(19) United States
(12) Patent Application Publication
(10) Pub. No.: US 2012/0301927 A1
(43) Pub. Date: Nov. 29, 2012

(54) Production Of Maltotetraose Syrup Using A Pseudomonas Saccharophila Maltotetraohydrolase Variant

(75) Inventors: Gang Duan, Shanghai (CN); Sung Ho Lee, North Liberty, IA (US); Ying Qian, Wuxi (CN); Rafael F. Sala, Mountain View, CA (US); Jayarama Shetty, Pleasanton, CA (US)

(73) Assignee: DANISCO US INC., Palo Alto, CA (US)

(21) Appl. No.: 13/263,419

(22) PCT Filed: Apr. 8, 2010
(86) PCT No.: PCT/US2010/030446

§ 371 Date: Feb. 24, 2012

Related U.S. Application Data
(60) Provisional application No. 61/168,437, filed on Apr. 10, 2009.

(51) Int. Cl. C12N 9/28; C12N 15/63; C12P 19/14; D06M 16/00; C12N 1/19; C12P 7/14; C12P 19/16; C12N 15/56; C12N 1/21

(52) U.S. Cl. 435/98; 435/202; 536/23.2; 435/320.1; 435/99; 435/252.31; 435/252.35; 435/252.33; 435/252.34; 435/254.2; 435/162; 435/263

(57) ABSTRACT

Variants of a Pseudomonas saccharophila G4-forming amylase (PS4) advantageously can catalyze high temperature saccharification to produce maltotetraose syrup from a starch liquefact or granular starch, e.g., derived from cornstarch. The PS4 variants are useful in a process of saccharification of starch that advantageously produces significant amounts of maltotetraose, which can be used downstream in a process of producing a maltotetraose syrup. In one embodiment, a thermostable PS4 variant is provided that can produce about 40% to about 60% by weight maltotetraose, based on total saccharide content.

### PRIORITY

The present application claims priority to U.S. Provisional Application Ser. No. 61/168,437 filed on Apr. 10, 2009, which is hereby incorporated by reference in its entirety.

### SEQUENCE LISTING

A Sequence Listing, comprising SEQ ID NOS: 1-4, is attached and is incorporated by reference in its entirety.

### FIELD OF THE INVENTION

A variant alpha-amylase from Pseudomonas saccharophila and nucleic acids encoding the same are useful for production of maltotetraose (G4) syrup, among other things.

### BACKGROUND

Maltotetraose (G4 or DP4) syrup is one of many commercially important products derived from enzymatic treatment of starch. The conversion of vegetable starches, especially cornstarch, to maltotetraose and lower sugars, such as glucose or maltose, is a rapidly expanding industry.

The current process consists of two sequential enzyme-catalyzed steps that result in the production of glucose or maltose. The first enzyme-catalyzed step is starch liquefaction. Typically, a starch suspension is gelatinized by rapid heating to about 85° C. or more. Alpha-amylases (EC 3.2.1.1) are used to degrade the viscous liquefact to maltodextrins. Alpha-amylases are endohydrolases that catalyze the random cleavage of internal α-1,4-D-glucosidic bonds. As alpha-amylases break down the starch, the viscosity decreases. Because liquefaction typically is conducted at high temperatures, thermostable alpha-amylases, such as an alpha-amylase from Bacillus sp., are preferred for this step.

A second enzyme-catalyzed saccharification step is required to break down the maltodextrins. Glucoamylases and/or maltogenic alpha-amylases commonly are used to catalyze the hydrolysis of non-reducing ends of the maltodextrins formed after liquefaction, releasing D-glucose, maltose and isomaltose. Debranching enzymes, such as pullulanases, can be used to aid saccharification. Saccharification typically takes place under acidic conditions at elevated temperatures, e.g., 60° C., pH 4.3.

G4 (also referred to as DP4) syrup has a number of advantageous properties compared to sucrose syrups. For example, partially replacing sucrose with G4 syrup in a food reduces the food's sweetness without affecting its taste or flavor. G4 syrup has high moisture retention in foods and exhibits less deleterious Maillard reaction products because of its lower glucose and maltose content. G4 syrup also has higher viscosity than sucrose, thus improving food texture. G4 syrup depresses the freezing point of water less than sucrose or high fructose syrup, so G4 syrup can better control the freezing points of frozen foods. After ingestion, G4 syrup also affects osmotic pressure less than sucrose. Together, these qualities make G4 syrup ideally suited as an ingredient in foods and medical products. G4 syrup is useful in other industries, as well. For example, G4 syrup imparts gloss and can be used advantageously as a paper sizer. See, e.g., Kimura et al., “Maltotetraose, a new saccharide of tertiary property,” Starch 42: 151-57 (1990).

Pseudomonas saccharophila expresses a useful G4-forming amylase. The P. saccharophila G4-forming amylase is variously known in the art as P. saccharophila maltotetraohydrolase, “Amy3A,” “PSA,” “SAS,” or “PS4.” SAS and PS4 are used interchangeably herein. A nucleotide sequence encoding PS4 has been determined See Zhou et al., “Nucleotide sequence of the maltotetraohydrolase gene from Pseudomonas saccharophila,” FEBS Lett. 255: 37-41 (1989); GenBank Acc. No. X16732. PS4 is expressed as a precursor protein with an N-terminal 21-residue signal peptide. The amino acid sequence of the PS4 precursor is set forth in SEQ ID NO: 1. The signal peptide is cleaved to form the mature form of PS4 containing 530 amino acid residues. The mature form has a catalytic domain at the N-terminus and a starch-binding domain at the C-terminus. The C-terminal starch binding domain of PS4 may be removed, leaving the catalytically active portion of PS4 having the amino acid sequence set forth in SEQ ID NO: 2. PS4 displays both endo- and exo-alpha-amylase activity. While endo-alpha-amylase activity is particularly useful for decreasing the viscosity of gelatinized starch, exo-alpha-amylase activity is particularly useful for breaking down maltodextrins to smaller saccharides, such as G4.

G4-forming amylases, such as the P. stutzeri G4-forming amylase, can be used in a continuous process of converting a liquefied starch to maltotetraose. In this process, the G4-forming amylase may be immobilized along with a pullulanase. See, e.g., Kimura et al., “Continuous production of maltotetraose using a dual immobilized enzyme system of maltotetraose-forming amylase and pullulanase,” Biotech. Bioeng'g 36: 790-96 (1990). The usefulness of the continuous reaction process is limited by the temperature-dependent half-life of the immobilized G4-forming amylase. See, id.

### SUMMARY

A Pseudomonas saccharophila maltotetraohydrolase (PS4) variant advantageously produces a significant amount of maltotetraose from either liquefied starch or other source of maltodextrins at a high temperature, e.g., about 60-70° C. The variant PS4 can be used to produce a maltotetraose syrup, among other things.

A starch processing composition comprising a PS4 variant is provided. The PS4 variant derives from a wild-type PS4 having an amino acid sequence of SEQ ID NO: 2 and has alpha-amylase activity. The PS4 variant may comprise a G233E amino acid substitution and up to 10, 11, 12, 13, 14, 15, 16, 17, 18, 19, 20, 21, 22, 23, or 24 additional amino acid deletions, additions, insertions, or substitutions compared to the amino acid sequence of SEQ ID NO: 2. Alternatively, the PS4 variant may have at least about 70%, about 80%, about 90%, or about 95% sequence identity to the amino acid sequence of SEQ ID NO: 2. In one embodiment, the PS4 variant has an amino-terminus methionine residue. In anther embodiment, the PS4 variant comprises a polypeptide sequence with up to 5, 6, 7, 8, 9, 10, 11, 12, 13, 14, or 15 additional amino acid substitutions compared to the amino acid sequence of SEQ ID NO: 2. The PS4 variant may comprise one or more following amino acid substitutions: N33Y, D34N, G70D, G121F, G134R, A141P, Y146G, I157L, S161A, L178F, A179T, S229P, H307K, A309P, or S334P. In one embodiment, the variant PS4 comprises the amino acid sequence of SEQ ID NO: 3 (i.e., SAS3). The variant PS4 may be isolated or purified.

In some embodiments, variants of PS4 have altered properties compared to wild-type PS4. For example, the variant PS4 may have an altered, e.g., higher, thermostability compared to wild-type PS4. The variant PS4 may have an altered, e.g., higher, pH stability compared to wild-type PS4. The pH stability may be more stable that the wild-type PS4 at a pH of about 5.0 to about 7.0. The variant may have more exo-alpha-amylase activity than wild-type PS4 or may have less endo-alpha-amylase activity than wild-type PS4. A starch processing composition may comprise any of the PS4 variants above.

Also provided is a method of making a saccharide (e.g., maltotetraose) syrup, comprising adding a PS4 variant or a composition comprising the variant to a starch liquefact and saccharifying the starch liquefact to form the saccharide syrup. The PS4 variant may be added to the starch liquefact in a range from about 0.001% by weight to about 0.1% by weight based on dissolved solids. In one embodiment, the variant is added to the starch liquefact in a range from about 0.0025% by weight to about 0.01% by weight based on dissolved solids. The units of concentration also are expressed herein as kg of variant PS4 per metric ton of dry solids (MTDS), where 1 kg/MTDS=0.1% by weight dissolved solids. The liquefied starch solution may be a slurry of liquefied starch at about 20-35% w/w dry solids. The starch may be obtained from corns, cobs, wheat, barley, rye, milo, sago, cassava, tapioca, sorghum, rice, peas, bean, banana, or potatoes. The starch liquefact may be saccharified at about 60° C. to about 65° C. The starch liquefact may be saccharified at about pH 5.0 to about pH 7.0. A pullulanase, isoamylase, pullulanase, protease, cellulase, hemicellulase, lipase, cutinase, or any combination thereof, may be added with the variant PS4 to the starch liquefact. In one embodiment, the saccharide syrup may be fermented to produce ethanol. The saccharide syrup produced by the method may comprise at least about 40%, about 45%, about 50%, about 55%, or about 60% by weight maltotetraose based on total saccharide content.

In another aspect a method of making a saccharide syrup, including adding a PS4 variant and an alpha-amylase to granular starch and hydrolyzing the granular starch to form the saccharide syrup is provided. In one embodiment the PS4 variant is added to the granular starch in a range from about 0.001% by weight to about 0.1% by weight based on dissolved solids. In another embodiment the PS4 variant is added to the granular starch in a range from about 0.0025% by weight to about 0.01% by weight based on dissolved solids. The granular starch can be obtained from starch from corns, cobs, wheat, barley, rye, milo, sago, cassava, tapioca, sorghum, rice, peas, bean, banana, or potatoes.

In a particular embodiment the granular starch is saccharified at about 60° C. to about 65° C. In another embodiment the granular starch is saccharified at about pH 5.0 to about pH 7.0. It is envisioned that the method can also include fermenting the saccharide syrup to produce ethanol.

In one embodiment the method includes a step of adding an enzyme having debranching activity to the granular starch. The enzyme having debranching activity can include but is not limited to an isoamylase, a pullulanase, an isopullulanase, a neopullulanase or any combination thereof. It is also envisioned that the method can optionally include a further step of adding a protease, a cellulase, a hemicellulase, a lipase, a cutinase, a pectate liase or any combination thereof to the granular starch.

In one embodiment the saccharide syrup includes at least about 40% by weight maltotetraose based on total saccharide content. Alternatively, the saccharide syrup includes at least about 45% by weight maltotetraose based on total saccharide content. In another embodiment the saccharide syrup includes at least about 50% by weight maltotetraose based on total saccharide content. In a further embodiment the saccharide syrup includes from about 45% by weight to about 60% by weight maltotetraose based on total saccharide content.

It is envisioned that the PS4 variant of the method can be immobilized.

In another aspect a method is provided for making IMO, including adding a) a PS4 variant, b) an alpha-amylase, and c) a transglucosidase to starch in the form of a starch liquefact or granular starch and saccharifying the starch to form IMO. It is envisioned that the IMO can be formed at an IMO number of at least 30, at least 40 and/or at least 45. In one embodiment the starch is obtained from corns, cobs, wheat, barley, rye, milo, sago, cassava, tapioca, sorghum, rice, peas, bean, banana, or potatoes.

Also provided is a textile desizing composition comprising a PS4 variant in an aqueous solution, and optionally with another enzyme. A method of desizing a textile comprises contacting the textile desizing composition with a textile for a time and under conditions sufficient to desize the textile.

### BRIEF DESCRIPTION OF THE DRAWINGS

The accompanying drawings are incorporated in and constitute a part of this specification and illustrate various embodiments. In the drawings below, “PS4” is replaced with the abbreviation “SAS.” The abbreviations refer to the same protein and are interchangeable.

FIG. 1 depicts an exemplary HPLC chromatogram of liquefied cornstarch saccharified using SAS3 (SEQ ID NO: 3). The chromatogram demonstrates formation of G4 (“DP4”) by the PS4 variant.

FIG. 2 depicts an HPLC chromatogram of a control sample of maltodextrin slurry under conditions of pH 6.5 at 60° C. for 18 hours. These reaction conditions are used in the experiments depicted in FIG. 3-FIG. 5.

FIG. 3 depicts an HPLC chromatogram of a maltodextrin slurry treated with SAS3 (SEQ ID NO: 3) at 0.025 kg/metric ton dry solids (MTDS) under the same conditions as used in FIG. 2.

FIG. 4 depicts an HPLC chromatogram of a maltodextrin slurry treated with SAS3 (SEQ ID NO: 3) at 0.05 kg/MTDS under the same conditions as used in FIG. 2.

FIG. 5 depicts an HPLC chromatogram of a maltodextrin slurry treated with SAS3 (SEQ ID NO: 3) at 0.1 kg/MTDS under the same conditions as used in FIG. 2.

FIG. 6 depicts DP1, DP2, DP3, DP4, and DP5+ accumulation (% total saccharides) as a function of time (hr) for a maltodextrin slurry treated with 0.007 kg/MTDS SAS3 (SEQ ID NO: 3) at pH 5.5, 60° C.

FIG. 7 depicts DP1, DP2, DP3, DP4, and DP5+ accumulation (% total saccharides) as a function of time (hr) for a maltodextrin slurry treated with 0.012 kg/MTDS SAS3 (SEQ ID NO: 3) under the same conditions as used in FIG. 6.

FIG. 8 depicts DP1, DP2, DP3, DP4, and DP5+ accumulation (% total saccharides) as a function of time (hr) for a maltodextrin slurry treated with 0.024 kg/MTDS SAS3 (SEQ ID NO: 3) under the same conditions as used in FIG. 6.

FIG. 9 depicts the percent accumulation of DP4 at various pHs for a maltodextrin slurry treated with 0.025 kg/MTDS SAS3 (SEQ ID NO: 3) at 60° C.

FIG. 10 depicts the percent accumulation of DP4 at various temperatures for a maltodextrin slurry treated with 0.025 kg/MTDS SAS3 (SEQ ID NO: 3) at pH 5.0.

FIG. 11 depicts the percent accumulation of DP4 at various concentrations of SAS3 (SEQ ID NO: 3) in a maltodextrin slurry at pH 5.0, 60° C.

FIG. 12 depicts the percent accumulation of DP4 at various concentrations of pullulanase in a maltodextrin slurry treated with 0.025 kg/MTDS SAS3 (SEQ ID NO: 3) at pH 5.0, 65° C.

FIG. 13 depicts the percent accumulation of DP4 at various substrate concentrations (% DS starch) for a maltodextrin slurry treated with 0.01 kg/MTDS SAS3 (SEQ ID NO: 3) and 1 kg/MTDS pullulanase at pH 5.0, 60° C.

### DETAILED DESCRIPTION

Variants of a Pseudomonas saccharophila G4-forming amylase (PS4) advantageously can catalyze high temperature saccharification to produce maltotetraose syrup from a starch liquefact, e.g., derived from cornstarch. The PS4 variants are useful in a process of saccharification of starch that advantageously produces significant amounts of maltotetraose, which can be used downstream in a process of producing a maltotetraose syrup. In one embodiment, a thermostable PS4 variant is provided that can produce about 40% to about 60% by weight maltotetraose, based on total saccharide content. PS4 may occasionally be referred to as SAS in the specification and figures. “PS4” and “SAS” are synonymous. As an example, “SAS3” in all occurrences refers to a PS4 variant.

### 1. DEFINITIONS AND ABBREVIATIONS

In accordance with this detailed description, the following abbreviations and definitions apply. It should be noted that as used herein, the singular forms “a,” “an,” and “the” include plural referents unless the context clearly dictates otherwise. Thus, for example, reference to “an enzyme” includes a plurality of such enzymes, and reference to “the formulation” includes reference to one or more formulations and equivalents thereof known to those skilled in the art, and so forth.

Unless defined otherwise, all technical and scientific terms used herein have the same meaning as commonly understood by one of ordinary skill in the art. The following terms are provided below.

### 1.1. Definitions

“Amylase” means an enzyme that is, among other things, capable of catalyzing the degradation of starch. An endo-acting amylase activity cleaves α-D-(1→4) O-glycosidic linkages within the starch molecule in a random fashion. In contrast, an exo-acting amylolytic activity cleaves a starch molecule from the non-reducing end of the substrate. “Endo-acting amylase activity,” “endo-activity,” “endo-specific activity,” and “endo-specificity” are synonymous, when the terms refer to PS4. The same is true for the corresponding terms for exo-activity. Useful alpha-amylases from Bacillus sp. include but are not limited to SPEZYME® FRED and SPEZYME® ALPHA (Danisco US Inc., Genencor Division).

A “variant,” or “variants” refers to either polypeptides or nucleic acids. The term “variant” may be used interchangeably with the term “mutant.” Variants include insertions, additions, deletions, substitutions, transversions, truncations, and/or inversions at one or more locations in the amino acid or nucleotide sequence, respectively. The phrases “variant polypeptide,” and “variant enzyme” mean a PS4 protein that has an amino acid sequence that has been modified from the amino acid sequence of a wild-type PS4. The variant polypeptides include a polypeptide having a certain percent, e.g., at least 70%, 75%, 80%, 85%, 90%, 95%, or 99% (or any integer value between these numbers), of sequence identity with the parent enzyme. Variant polypeptides particularly may have a certain number of amino acid additions, deletions, or substitutions compared to the wild-type PS4. For example, PS4 variants may have 1 to 25, e.g., 1-5, 1-10, 1-15, or 1-20, amino acid additions deletions, or substitutions.

As used herein, “parent enzymes,” “parent sequence,” “parent polypeptide,” “wild-type PS4,” and “parent polypeptides” mean enzymes and polypeptides from which the variant polypeptides are based, e.g., the PS4 of SEQ ID NO: 1. A “parent nucleic acid” means a nucleic acid sequence encoding the parent polypeptide. A “wild-type” PS4 occurs naturally and includes naturally occurring allelic variants of the PS4 of SEQ ID NO: 1. The signal sequence of a “variant” may be the same or may differ from the wild-type PS4. A variant may be expressed as a fusion protein containing a heterologous polypeptide. For example, the variant can comprise a signal peptide of another protein or a sequence designed to aid identification or purification of the expressed fusion protein, such as a His-Tag sequence.

“Variant nucleic acids” can include sequences that are complementary to sequences that are capable of hybridizing to the nucleotide sequences presented herein. For example, a variant sequence is complementary to sequences capable of hybridizing under stringent conditions, e.g., 50° C. and 0.2×SSC (1×SSC=0.15 M NaCl, 0.015 M sodium citrate, pH 7.0), to the nucleotide sequences presented herein. More particularly, the term variant encompasses sequences that are complementary to sequences that are capable of hybridizing under highly stringent conditions, e.g., 65° C. and 0.1×SSC, to the nucleotide sequences presented herein. The melting point (Tm) of a variant nucleic acid may be about 1, 2, or 3° C. lower than the Tm of the wild-type nucleic acid. The variant nucleic acids include a polynucleotide having a certain percent, e.g., at least 70%, 75%, 80%, 85%, 90%, 95%, or 99%, of sequence identity with the nucleic acid encoding the parent enzyme.

To describe the various variants, the following nomenclature will be adopted for ease of reference. Where the substitution includes a number and a letter, e.g., 141P, then this refers to {position according to the numbering system/substituted amino acid}. Accordingly, for example, the substitution of an amino acid to proline in position 141 is designated as 141P. Where the substitution includes a letter, a number, and a letter, e.g., A141P, then this refers to {original amino acid/position according to the numbering system/substituted amino acid}. Accordingly, for example, the substitution of alanine with proline in position 141 is designated as A141P.

Where two or more substitutions are possible at a particular position, this will be designated by contiguous letters, which may optionally be separated by slash marks “/”, e.g., G303ED or G303E/D.

Sequence identity is determined using standard techniques known in the art (see e.g., Smith and Waterman, Adv. Appl. Math. 2: 482 (1981); Needleman and Wunsch, J. Mol. Biol. 48: 443 (1970); Pearson and Lipman, Proc. Natl. Acad. Sci. USA 85: 2444 (1988); programs such as GAP, BESTHT, FASTA, and TFASTA in the Wisconsin Genetics Software Package (Genetics Computer Group, Madison, Wis.); and Devereux et al., Nucleic Acid Res., 12: 387-395 (1984)).

The “percent (%) nucleic acid sequence identity” or “percent (%) amino acid sequence identity” is defined as the percentage of nucleotide residues or amino acid residues in a candidate sequence that are identical with the nucleotide residues or amino acid residues of the starting sequence. The sequence identity can be measured over the entire length of the starting sequence.

“Sequence identity” is determined herein by the method of sequence alignment. For the purpose of the present disclosure, the alignment method is BLAST described by Altschul et al., (Altschul et al., J. Mol. Biol. 215: 403-410 (1990); and Karlin et al, Proc. Natl. Acad. Sci. USA 90: 5873-5787 (1993)). A particularly useful BLAST program is the WU-BLAST-2 program (see Altschul et al, Meth. Enzymol. 266: 460-480 (1996)). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched. However, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

As used herein, the term “expression” refers to the process by which a polypeptide is produced based on the nucleic acid sequence of a gene. The process includes both transcription and translation.

The term “isolated” refers to a material that is removed from the natural environment if it is naturally occurring.

A “purified” protein or enzyme refers to a protein that is at least partially purified to homogeneity. In some embodiments, a purified protein or enzyme is more than 10% pure, optionally more than 20% pure, and optionally more than 30% pure, as determined by SDS-PAGE. Further aspects of the disclosure encompass the protein in a highly purified form (i.e., more than 40% pure, more than 60% pure, more than 80% pure, more than 90% pure, more than 95% pure, more than 97% pure, and even more than 99% pure), as determined by SDS-PAGE.

“Thermostable” or “thermostability” means the enzyme retains activity after exposure to elevated temperatures. The thermostability of an enzyme is measured by its half-life (t_{1/2}), where half of the enzyme activity is lost by the half-life. The half-life value is calculated under defined conditions by measuring the residual amylase activity. To determine the half-life of the enzyme, the sample is heated to the test temperature for 1-10 min, and activity is measured using a standard assay for PS4 activity, such as the Betamyl® assay (Megazyme, Ireland).

As used herein, “optimum pH” means the pH at which PS4 or a PS4 variant displays the activity in a standard assay for PS4 activity, measured over a range of pH's.

As used herein, “amino acid sequence” is synonymous with the term “polypeptide” and/or the term “protein.” In some instances, the term “amino acid sequence” is synonymous with the term “peptide”; in some instances, the term “amino acid sequence” is synonymous with the term “enzyme.”

As used herein, “nucleotide sequence” or “nucleic acid sequence” refers to an oligonucleotide sequence or polynucleotide sequence and variants, homologues, fragments and derivatives thereof. The nucleotide sequence may be of genomic, synthetic or recombinant origin and may be double-stranded or single-stranded, whether representing the sense or anti-sense strand. As used herein, the term “nucleotide sequence” includes genomic DNA, cDNA, synthetic DNA, and RNA.

“Homologue” means an entity having a certain degree of identity or “homology” with the subject amino acid sequences and the subject nucleotide sequences. A “homologous sequence” includes a polynucleotide or a polypeptide having a certain percent, e.g., 70%, 75%, 80%, 85%, 90%, 95%, or 99% (or any integer value in between), of sequence identity with another sequence. Percent identity means that, when aligned, that percentage of bases or amino acid residues are the same when comparing the two sequences Amino acid sequences are not identical, where an amino acid is substituted, deleted, or added compared to the subject sequence. The percent sequence identity typically is measured with respect to the mature sequence of the subject protein, i.e., following removal of a signal sequence, for example. Typically, homologues will comprise the same active site residues as the subject amino acid sequence. Homologues also retain amylase activity, although the homologue may have different enzymatic properties than the wild-type PS4.

As used herein, “hybridization” includes the process by which a strand of nucleic acid joins with a complementary strand through base pairing, as well as the process of amplification as carried out in polymerase chain reaction (PCR) technologies. The variant nucleic acid may exist as single- or double-stranded DNA or RNA, an RNA/DNA heteroduplex or an RNA/DNA copolymer. As used herein, “copolymer” refers to a single nucleic acid strand that comprises both ribonucleotides and deoxyribonucleotides. The variant nucleic acid may be codon-optimized to further increase expression.

As used herein, a “synthetic” compound is produced by in vitro chemical or enzymatic synthesis. It includes, but is not limited to, variant nucleic acids made with optimal codon usage for host organisms, such as a yeast cell host or other expression hosts of choice.

As used herein, “transformed cell” includes cells, including both bacterial and fungal cells, which have been transformed by use of recombinant DNA techniques. Transformation typically occurs by insertion of one or more nucleotide sequences into a cell. The inserted nucleotide sequence may be a heterologous nucleotide sequence, i.e., is a sequence that is not natural to the cell that is to be transformed, such as a fusion protein.

As used herein, “operably linked” means that the described components are in a relationship permitting them to function in their intended manner. For example, a regulatory sequence operably linked to a coding sequence is ligated in such a way that expression of the coding sequence is achieved under condition compatible with the control sequences.

As used herein, “biologically active” refers to a sequence having a similar structural, regulatory or biochemical function as the naturally occurring sequence, although not necessarily to the same degree.

As used herein the term “starch” refers to any material comprised of the complex polysaccharide carbohydrates of plants, such as corn, comprised of amylose and amylopectin with the formula (C₆H₁₀O₅)ₓ, where X can be any number. The term “granular starch” refers to raw, i.e., uncooked starch, e.g., starch that has not been subject to gelatinization.

The term “liquefaction” refers to the stage in starch conversion in which gelatinized starch is hydrolyzed to give low molecular weight soluble dextrins, i.e. polysaccharides. As used herein the term “saccharification” refers to enzymatic conversion of starch, liquefied starch, or maltodextrins to saccharides, e.g., glucose. The term “degree of polymerization” (DP) refers to the number (n) of anhydroglucopyranose units in a given saccharide. Examples of DP1 are the monosaccharides glucose and fructose. Examples of DP2 are the disaccharides maltose and sucrose. An example of DP4, as used herein, is maltotetraose (G4).

As used herein, the terms “dry solids content” or alternatively, “dissolved solids” (ds) refers to the total solids of a slurry or solution in a dry weight percent basis. The term “slurry” refers to an aqueous mixture containing insoluble solids.

The phrase “simultaneous saccharification and fermentation (SSF)” refers to a process in the production of biochemicals in which a microbial organism, such as an ethanol producing microorganism and at least one enzyme, such as PS4 or a variant thereof, are present during the same process step. SSF refers to the contemporaneous hydrolysis of granular starch substrates to saccharides and the fermentation of the saccharides into alcohol, for example, in the same reactor vessel.

As used herein “ethanologenic microorganism” refers to a microorganism with the ability to convert a sugar or oligosaccharide to ethanol.

### 1.2. Abbreviations

The following abbreviations apply unless indicated otherwise:
ADA azodicarbonamide
cDNA complementary DNA
CGTase cyclodextrin glucanotransferase
DE dextrose equivalence
DEAE diethylamino ethanol
DNA deoxyribonucleic acid
DPn degree of polymerization with n glucose subunits
ds dry solids or dissolved solids
EC enzyme commission for enzyme classification
FGSC Fungal Genetics Stock Center
G223E glycine (G) residue at position 223 of SEQ ID NO: 2 is replaced with a glutamic acid (E) residue, where amino acids are designated by single letter abbreviations commonly known in the art
G4 maltotetraose
GRAS generally recognized as safe
HPLC High Performance Liquid Chromatography
IMO isomalto-oligosaccharide
LU Lipase Units, a measure of phospholipase activity per unit mass of enzyme
MTDS Metric tons dry solids
mRNA messenger ribonucleic acid
PCR polymerase chain reaction
PEG polyethyleneglycol
ppm parts per million
PS4 P. saccharophila G4-forming amylase
RO water Reverse osmosis water
RT-PCR reverse transcriptase polymerase chain reaction
SAS P. saccharophila G4-forming amylase
SDS-PAGE sodium dodecyl sulfate-polyacrylamide gel electrophoresis
1×SSC 0.15 M NaCl, 0.015 M sodium citrate, pH 7.0
SSF simultaneous saccharification and fermentation
t_{1/2} half life
Tm melting temperature (° C.) at which 50% of the subject protein is melted
w/v weight/volume
w/w weight/weight

### 2. Pseudomonas saccharophila ALPHA-AMYLASE (PS4) VARIANTS

An isolated and/or purified polypeptide comprising a variant PS4 is provided. In one embodiment, the variant PS4 is a mature form of the polypeptide, wherein the 21 amino acid leader sequence is cleaved, so that the N-terminus of the polypeptide begins at the aspartic acid (D) residue at position 22 of SEQ ID NO: 1. Variants of PS4 include a PS4 in which the C-terminal starch binding domain is removed. A representative amino acid sequence of a mature PS4 in which the starch biding domain is removed is set forth in SEQ ID NO: 2. Other PS4 variants include variants wherein between one and about 25 amino acid residues have been added or deleted with respect to wild-type PS4 or the PS4 of SEQ ID NO: 2. In one aspect, the PS4 variant has the amino acid sequence shown in SEQ ID NO: 2, wherein any number between one and about 25 amino acids have been substituted. In another aspect, a PS4 variant may have one or more amino acids added to the N-terminus of the PS4 of SEQ ID NO: 2, and the same variant may include between one and about 25 amino acids that have been substituted in the same sequence. A representative embodiment of these variants is set forth in SEQ ID NO: 3.

In another aspect, the PS4 variant has the sequence of wild-type PS4, wherein any number between one and about 25 amino acids have been substituted. Representative examples of PS4 variants having single amino acid substitutions are shown in TABLE 5. An example of a PS4 variant having combinations of amino acid substitutions is shown in TABLE 6. TABLE 6 depicts various amino acids that have been modified to form the sequence of SEQ ID NO: 3 (SAS3). In addition to the amino acid residue modifications listed in TABLES 5-6, additional specific PS4 residues that may be modified include A3, S44, A93, G103, V109, G172, A211, G265, N302, G313, and G342. PS4 variants may have various combinations of the amino acid substitutions disclosed herein. A process of using a PS4 variant may comprise the use of a single PS4 variant or a combination, or blend, of PS4 variants.

In one embodiment, the PS4 variant comprises an N-terminal methionine. The addition of a methionine at the amino terminus of the polypeptide may increase fermentation yields, for example.

PS4 variants may be particularly useful in a saccharification process that favors formation of maltotetraose. For example, a saccharide syrup can be formed comprising at least about 40% by weight maltotetraose based on dissolved solids, i.e. based on total saccharide content. In a typical embodiment, a saccharide syrup can be formed comprising at least about 45% by weight maltotetraose based on dissolved solids, i.e. based on total saccharide content. In another typical embodiment, a saccharide syrup can be formed comprising at least about 50% by weight maltotetraose based on dissolved solids, i.e. based on total saccharide content. In yet another typical embodiment, the saccharide syrup comprises from about 45% by weight to about 60% by weight maltotetraose based on dissolved solids, i.e. based on total saccharide content.

A representative PS4 variant for formation of maltotetraose is SAS3, set forth in SEQ ID NO: 3. This variant has sixteen (16) substitutions that maintain or increase thermostability and pH stability compared to wild-type PS4: N33Y, D34N, G70D, G121F, G134R, A141P, Y146G, I157L, S161A, L178F, A179T, G223E, S229P, H307K, A309P, and S334P. In addition, this variant includes a methionine residue added to the N-terminus In one embodiment, the PS4 variant comprises one or more of the following substitutions: N33Y, D34N, G70D, G121F, G134R, A141P, Y146G, I157L, S161A, L178F, A179T, S229P, H307K, A309P, or S334P. Additional amino acid substitutions can be made, for example: G121D, G223A, H272Q, G303E, and H307L.

Other particularly useful variants include those in which residues affecting substrate binding are substituted. PS4 residues involved in substrate binding include W66, I157, E160, S161, R196, W221, K222, H307, and W308. Substitutions of residues that affect substrate binding may affect the relative degree of endo- or exo-activity of the PS4 variant. A substitution that increases exo-activity, for example, advantageously promotes the formation of DP4 and DP3 saccharides. Representative examples of mutations affecting substrate binding include E160G, E160P, E160F, E160R, E1605, E160L, W66S, R196V, R196H, R196P, H307L, H307K, W221A, W308A, W3085, W308L, W3085, and K222T. These and additional variants of PS4 are described in U.S. Ser. No. 12/318,513, filed Dec. 30, 2008, which is incorporated herein by reference in its entirety.

A contemplated PS4 variant may have at least about 70%, about 75%, about 80%, about 85%, about 90%, about 95%, about 98%, or about 99% sequence identity to the naturally occurring PS4 having an amino acid sequence of SEQ ID NO: 2. Moreover, the PS4 variant may display one or more altered properties compared to the PS4 having an amino acid sequence of SEQ ID NO: 2. Altered properties may include altered thermostability, altered stability at a given pH range, altered exo-alpha-amylase activity, or altered endo-alpha-amylase activity. The PS4 variant may display an improved thermostability and/or improved stability at a pH of about 5.0 to about 7.0 compared to the PS4 having an amino acid sequence of SEQ ID NO: 2. The PS4 variant may display an increased exo-alpha-amylase activity or an decreased endo-alpha-amylase activity compared to the PS4 having an amino acid sequence of SEQ ID NO: 2.

Nucleic acids encoding the polypeptides above also are provided. In one embodiment, a nucleic acid encoding a PS4 variant is a cDNA encoding the protein of SEQ ID NO: 2, comprising a codon modification that encodes a substituted amino acid. For example, the cDNA may have the corresponding sequence of the native mRNA, set forth in SEQ ID NO: 4. See GenBank Acc. No. X16732. As is well understood by one skilled in the art, the genetic code is degenerate, meaning that multiple codons in some cases may encode the same amino acid. Nucleic acids include genomic DNA, mRNA, and cDNA that encodes a PS4 variant.

### 2.1. PS4 Variant Characterization

Enzyme variants can be characterized by their nucleic acid and primary polypeptide sequences, by three dimensional structural modeling, and/or by their specific activity. Additional characteristics of the PS4 variant include altered stability, optimal pH, oxidation stability, ratio of exo-amylase to endo-amylase activity, and thermostability, for example. Levels of expression and enzyme activity can be assessed using standard assays known to the artisan skilled in this field. In another aspect, variants demonstrate improved performance characteristics relative to the wild-type enzyme, such as improved stability at high temperatures, e.g., about 60-70° C. PS4 variants are advantageous for use for saccharification or other processes that require elevated temperatures. For example, a thermostable PS4 variant can degrade starch at temperatures of about 55° C. to about 85° C. or more.

An expression characteristic means an altered level of expression of the variant, when the variant is produced in a particular host cell. Expression generally relates to the amount of active variant that is recoverable from a fermentation broth using standard techniques known in this art over a given amount of time. Expression also can relate to the amount or rate of variant produced within the host cell or secreted by the host cell. Expression also can relate to the rate of translation of the mRNA encoding the variant enzyme.

A nucleic acid complementary to a nucleic acid encoding any of the PS4 variants set forth herein is provided. Additionally, a nucleic acid capable of hybridizing to the complement is provided. In another embodiment, the sequence for use in the methods and compositions described here is a synthetic sequence. It includes, but is not limited to, sequences made with optimal codon usage for expression in host organisms, such as yeast or bacteria.

### 3. PRODUCTION OF PS4 Variants

The PS4 variants provided herein may be produced synthetically or through recombinant expression in a host cell, according to procedures well known in the art. The expressed PS4 variant optionally is isolated prior to use. In another embodiment, the PS4 variant is purified following expression. Leader or signal sequences can be cleaved. Methods of genetic modification and recombinant production of PS4 variants are described, for example, in U.S. Pat. Nos. 7,371,552, 7,166,453; 6,890,572; and 6,667,065; and U.S. Published Application Nos. 2007/0141693; 2007/0072270; 2007/0020731; 2007/0020727; 2006/0073583; 2006/0019347; 2006/0018997; 2006/0008890; 2006/0008888; and 2005/0137111. The relevant teachings of these disclosures, including PS4-encoding polynucleotide sequences, primers, vectors, selection methods, host cells, purification and reconstitution of expressed PS4 variants, and characterization of PS4 variants, including useful buffers, pH ranges, Ca²⁺ concentrations, substrate concentrations and enzyme concentrations for enzymatic assays, are herein incorporated by reference.

In another embodiment, suitable host cells include a Gram positive bacterium selected from the group consisting of Bacillus subtilis, B. licheniformis, B. lentus, B. brevis, B. stearothermophilus, B. alkalophilus, B. amyloliquefaciens, B. coagulans, B. circulans, B. lautus, B. thuringiensis, Streptomyces lividans, or S. murinus; or a Gram negative bacterium, wherein said Gram negative bacterium is Escherichia coli or a Pseudomonas species. In one embodiment, the host cell is B. subtilus, and the expressed protein is engineered to comprise a B. subtilus signal sequence, as set forth in further detail below.

In some embodiments, a host cell is genetically engineered to express an PS4 variant with an amino acid sequence having at least about 70%, about 75%, about 80%, about 85%, about 90%, about 95%, about 96%, about 97%, about 98% or about 99% identity with the wild-type PS4. In some embodiments, the polynucleotide encoding a PS4 variant will have a nucleic acid sequence encoding the protein of SEQ ID NO: 2 or a nucleic acid sequence having at least about 70%, about 75%, about 80%, about 85%, about 90%, about 95%, about 96%, about 97%, about 98% or about 99% sequence identity with a nucleic acid encoding the protein of SEQ ID NO: 2. In one embodiment, the nucleic acid sequence has at least about 70%, about 75%, about 80%, about 85%, about 90%, about 95%, about 96%, about 97%, about 98% or about 99% sequence identity to the nucleic acid of SEQ ID NO: 4.

### 3.1. Vectors

In some embodiments, a DNA construct comprising a nucleic acid encoding a PS4 variant is transferred to a host cell in an expression vector that comprises regulatory sequences operably linked to a PS4 encoding sequence. The vector may be any vector that can be integrated into a fungal host cell genome and replicated when introduced into a host cell. The FGSC Catalogue of Strains, University of Missouri, lists suitable vectors. Additional examples of suitable expression and/or integration vectors are provided in Sambrook et al., MOLECULAR CLONING: A LABORATORY MANUAL, 3^{rd} ed., Cold Spring Harbor Laboratory Press, Cold Spring Harbor, N.Y. (2001); Bennett et al., MORE GENE MANIPULATIONS IN FUNGI, Academic Press, San Diego (1991), pp. 396-428; and U.S. Pat. No. 5,874,276. Exemplary vectors include pFB6, pBR322, PUC18, pUC100 and pENTR/D, pDON™201, pDONR™221, pENTR™, pGEM®3Z and pGEM®4Z. Exemplary for use in bacterial cells include pBR322 and pUC19, which permit replication in E. coli, and pE194, for example, which permits replication in Bacillus.

In some embodiments, a nucleic acid encoding a PS4 variant is operably linked to a suitable promoter, which allows transcription in the host cell. The promoter may be derived from genes encoding proteins either homologous or heterologous to the host cell. Suitable non-limiting examples of promoters include cbh1, cbh2, egl1, and egl2 promoters. In one embodiment, the promoter is one that is native to the host cell. For example, when P. saccharophila is the host, the promoter is a native P. saccharophila promoter. An “inducible promoter” is a promoter that is active under environmental or developmental regulation. In another embodiment, the promoter is one that is heterologous to the host cell.

In some embodiments, the coding sequence is operably linked to a signal sequence. The DNA encoding the signal sequence may be the DNA sequence naturally associated with the PS4 nucleic acid to be expressed. In other embodiments, the DNA encoding the signal sequence is replaced with a nucleotide sequence encoding a signal sequence from a species other than P. saccharophila. In this embodiment, the polynucleotide that encodes the signal sequence is immediately upstream and in-frame of the polynucleotide that encodes the polypeptide. The signal sequence may be selected from the same species as the host cell. In one non-limiting example, the signal sequence is a cyclodextrin glucanotransferase (CGTase; EC 2.4.1.19) signal sequence from Bacillus sp., and the PS4 variant is expressed in a B. subtilus host cell. A methionine residue may be added to the N-terminus of the signal sequence.

In additional embodiments, a signal sequence and a promoter sequence comprising a DNA construct or vector to be introduced into a fungal host cell are derived from the same source. In some embodiments, the expression vector also includes a termination sequence. In one embodiment, the termination sequence and the promoter sequence are derived from the same source. In another embodiment, the termination sequence is homologous to the host cell.

In some embodiments, an expression vector includes a selectable marker. Examples of suitable selectable markers include those that confer resistance to antimicrobial agents, e.g., hygromycin or phleomycin. Nutritional selective markers also are suitable and include amdS, argB, and pyr4. In one embodiment, the selective marker is the amdS gene, which encodes the enzyme acetamidase; it allows transformed cells to grow on acetamide as a nitrogen source. The use of an A. nidulans amdS gene as a selective marker is described in Kelley et al., EMBO J. 4: 475-479 (1985) and Penttila et al., Gene 61: 155-164 (1987).

A suitable expression vector comprising a DNA construct with a polynucleotide encoding a PS4 variant may be any vector that is capable of replicating autonomously in a given host organism or integrating into the DNA of the host. In some embodiments, the expression vector is a plasmid. In some embodiments, two types of expression vectors for obtaining expression of genes are contemplated. The first expression vector comprises DNA sequences in which the promoter, PS4 coding region, and terminator all originate from the gene to be expressed. In some embodiments, gene truncation is obtained by deleting undesired DNA sequences, e.g., DNA encoding the C-terminal starch binding domain, to leave the domain to be expressed under control of its own transcriptional and translational regulatory sequences. The second type of expression vector is preassembled and contains sequences required for high-level transcription and a selectable marker. In some embodiments, the coding region for a PS4 gene or part thereof is inserted into this general-purpose expression vector, such that it is under the transcriptional control of the expression construct promoter and terminator sequences. In some embodiments, genes or part thereof are inserted downstream of the strong cbh1 promoter. In some embodiments, C-terminal truncation of expressed PS4 variant is contemplated. For example, C-terminal truncation of alpha-amylases is described in Ohdan et al., Applied and Environ. Microbiol. 65: 4652-4658 (1999).

### 3.2. Transformation, Expression and Culture of Host Cells

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Ausubel et al. (1987), supra, chapter 9; Sambrook et al. (2001), supra; and Campbell et al., Curr. Genet. 16: 53-56 (1989). The expression of heterologous protein in Trichoderma is described, for example, in U.S. Pat. No. 6,022,725; U.S. Pat. No. 6,268,328; Harkki et al., Enzyme Microb. Technol. 13: 227-233 (1991); Harkki et al., BioTechnol. 7: 596-603 (1989); EP 244,234; and EP 215,594. In one embodiment, genetically stable transformants are constructed with vector systems whereby the nucleic acid encoding a PS4 variant is stably integrated into a host cell chromosome. Transformants are then purified by known techniques.

In one non-limiting example, stable transformants including an amdS marker are distinguished from unstable transformants by their faster growth rate and the formation of circular colonies with a smooth, rather than ragged outline on solid culture medium containing acetamide. Additionally, in some cases a further test of stability is conducted by growing the transformants on solid non-selective medium, e.g., a medium that lacks acetamide, harvesting spores from this culture medium and determining the percentage of these spores that subsequently germinate and grow on selective medium containing acetamide. Other methods known in the art may be used to select transformants.

### 3.3. Identification of PS4 Activity

To evaluate the expression of a PS4 variant in a host cell, assays can measure the expressed protein, corresponding mRNA, or alpha-amylase activity. For example, suitable assays include Northern and Southern blotting, RT-PCR (reverse transcriptase polymerase chain reaction), and in situ hybridization, using an appropriately labeled hybridizing probe. Suitable assays also include measuring PS4 activity in a sample. Suitable assays of the exo-activity of the PS4 variant include, but are not limited to, the Betamyl® assay (Megazyme, Ireland). Suitable assays of the endo-activity of the PS4 variant include, but are not limited to, the Phadebas blue assay (Magle Life Sciences). Assays also include HPLC analysis of saccharide syrup prepared in the presence of the PS4 variant. HPLC, for example, can be used to measure amylase activity by separating DP4 saccharides from other saccharides in the reaction mixture.

### 3.4. Methods for Purifying PS4

In general, a PS4 variant produced in cell culture is secreted into the medium and may be purified or isolated, e.g., by removing unwanted components from the cell culture medium. In some cases, a PS4 variant may be recovered from a cell lysate. In such cases, the enzyme is purified from the cells in which it was produced using techniques routinely employed by those of skill in the art. Examples include, but are not limited to, affinity chromatography, ion-exchange chromatographic methods, including high resolution ion-exchange including HPLC on sulfonated styrene-divinylbenzene ion-exchange resin, hydrophobic interaction chromatography, two-phase partitioning, ethanol precipitation, reverse phase HPLC, chromatography on silica or on a cation-exchange resin, such as DEAE, chromatofocusing, SDS-PAGE, ammonium sulfate precipitation, gel permeation chromatography (GPC), and gel filtration (size exclusion chromatography) using Sephadex G-75, for example.

### 4. COMPOSITIONS AND USES OF PS4 Variants

A PS4 variant produced and purified by the methods described above is useful for a variety of industrial applications. In one embodiment, the PS4 variant is useful in a starch conversion process, particularly in a saccharification process of a starch, e.g., corn starch, wheat starch, or barley starch. The desired end-product may be any product that may be produced by the enzymatic conversion of the starch substrate. For example, the desired product may be a syrup rich in maltotetraose, which can be used in the manufacture of foods, particularly frozen foods, or as a component in medicaments.

The desirability of using a particular PS4 variant will depend on the overall properties displayed by the PS4 variant relative to the requirements of a particular application. For example, PS4 variants useful for a starch conversion process may have substantial endo-amylase activity compared to wild-type PS4, and/or have a lower exo- to endo-amylase activity compared to wild-type PS4. Such PS4 variants may be particularly useful in a process where internal cleavage of complex branching saccharides in useful in lowering the viscosity of the substrate. Useful PS4 variants include those with more or less exo-amylase activity than the wild-type PS4, depending on the application. Compositions may include one or a combination of PS4 variants, each of which may display a different set of properties.

### 4.1. Preparation of Starch Substrates

Methods to prepare starch substrates are well known in the art. For example, a useful starch substrate may be obtained from tubers, roots, stems, legumes, cereals or whole grain. More specifically, the granular starch comes from plants that produce high amounts of starch. For example, granular starch may be obtained from corns, cobs, wheat, barley, rye, milo, sago, cassava, tapioca, sorghum, rice, peas, bean, banana, or potatoes. Corn contains about 60-68% starch; barley contains about 55-65% starch; millet contains about 75-80% starch; wheat contains about 60-65% starch; and polished rice contains 70-72% starch. Specifically contemplated starch substrates are cornstarch, wheat starch, and barley starch. The starch from a grain may be ground or whole and includes corn solids, such as kernels, bran and/or cobs. The starch may be highly refined raw starch or feedstock from starch refinery processes. Various starches also are commercially available. For example, cornstarch is available from Cerestar, Sigma, and Katayama Chemical Industry Co. (Japan); wheat starch is available from Sigma; sweet potato starch is available from Wako Pure Chemical Industry Co. (Japan); and potato starch is available from Nakaari Chemical Pharmaceutical Co. (Japan).

Maltodextrins are useful as starch substrates in embodiments of the present invention. Maltodextrins comprise starch hydrolysis products having about 20 or fewer dextrose (glucose) units. Typical commercial maltodextrins contain mixtures of polysaccharides including from about three to about nineteen linked dextrose units. Maltodextrins are defined by the FDA as products having a dextrose equivalence (DE) of less than 20. They are generally recognized as safe (GRAS) food ingredients for human consumption. Dextrose equivalence (DE) is a measure of reducing power compared to a dextrose (glucose) standard of 100. The higher the DE, the greater the extent of starch depolymerization, resulting in a smaller average polymer (polysaccharide) size, and the greater the sweetness. A particularly useful maltodextrin is MALTRIN® M040 obtained from cornstarch, available from Grain Processing Corp. (Muscatine, Iowa): DE 4.0-7.0; bulk density 0.51 g/cc; measured water content 6.38% by weight.

The starch substrate can be a crude starch from milled whole grain, which contains non-starch fractions, e.g., germ residues and fibers. Milling may comprise either wet milling or dry milling. In wet milling, whole grain is soaked in water or dilute acid to separate the grain into its component parts, e.g., starch, protein, germ, oil, kernel fibers. Wet milling efficiently separates the germ and meal (i.e., starch granules and protein) and is especially suitable for production of syrups. In dry milling, whole kernels are ground into a fine powder and processed without fractionating the grain into its component parts. Dry milled grain thus will comprise significant amounts of non-starch carbohydrate compounds, in addition to starch. Alternatively, the starch to be processed may be a highly refined starch quality, for example, at least 90%, at least 95%, at least 97%, or at least 99.5% pure.

### 4.2. Saccharification of Liquefied Starch

As used herein, the term “liquefaction” or “liquefy” means a process by which starch is converted to less viscous and shorter chain dextrins. This process involves gelatinization of starch simultaneously with or followed by the addition of a PS4 variant. A thermostable PS4 variant is typically used for this application. Additional liquefaction-inducing enzymes optionally may be added.

In some embodiments, the starch or maltodextrin substrate prepared as described above is slurried with water. The starch or maltodextrin slurry may contain starch as a weight percent of dry solids of about 10-55%, about 20-45%, about 30-45%, about 30-40%, or optionally about 30-35%. Alpha-amylases, e.g., bacterial alpha-amylases, including Bacillus alpha-amylases, may be supplied, at about 1500 units per kg dry matter of starch, for example. To optimize alpha-amylase stability and activity, the pH of the slurry may be adjusted to the optimal pH for the α-amylase. Other alpha-amylases may be added and may require different optimal conditions. Bacterial alpha-amylase remaining in the slurry following liquefaction may be deactivated by lowering pH in a subsequent reaction step or by removing calcium from the slurry.

The slurry of starch may be pumped continuously through a jet cooker, which is steam heated from about 85° C. to up to 105° C. Gelatinization occurs very rapidly under these conditions, and the enzymatic activity, combined with the significant shear forces, begins the hydrolysis of the starch substrate. The residence time in the jet cooker is very brief. The partly gelatinized starch may be passed into a series of holding tubes maintained at about 85-105° C. and held for about 5 min. to complete the gelatinization process. These tanks may contain baffles to discourage back mixing. As used herein, the term “secondary liquefaction” refers the liquefaction step subsequent to primary liquefaction, when the slurry is allowed to cool to room temperature. This cooling step can be about 30 minutes to about 180 minutes, e.g. about 90 minutes to 120 minutes.

PS4 variant can be added to the liquefied starch obtained by the process above or to a maltodextrin slurry at about 0.01 to about 1.0 kg/MTDS. 1 kg/MTDS=0.1% by weight dissolved solids. In one embodiment, a PS4 variant can be added to a liquefied starch or maltodextrin slurry at a treatment level in a range from about 0.001% by weight to about 0.01% by weight based on dissolved solids. In a typical embodiment, a PS4 variant can be added to a liquefied starch or maltodextrin slurry at a treatment level in a range from about 0.0025% by weight to about 0.01% by weight based on dissolved solids. In one embodiment, the PS4 variant is immobilized, and the liquefied starch or maltodextrin substrate is passed over the immobilized PS4 variant and converted to product in a continuous reaction. In this embodiment, the PS4 variant may be immobilized with additional enzymes, such as a pullulanase.

The production of maltotetraose may further comprise contacting the liquefied starch or other source of maltodextrins with an isoamylase, a protease, a cellulase, a hemicellulase, a lipase, a cutinase, or any combination thereof.

### 5. TEXTILE DESIZING COMPOSITIONS AND USE

Also contemplated are compositions and methods of treating fabrics (e.g., to desize a textile) using PS4 variant. Fabric-treating methods are well known in the art (see, e.g., U.S. Pat. No. 6,077,316). For example, in one aspect, the feel and appearance of a fabric is improved by a method comprising contacting the fabric with a PS4 variant in a solution. In one aspect, the fabric is treated with the solution under pressure.

In one aspect, a PS4 variant is applied during or after the weaving of a textile, or during the desizing stage, or one or more additional fabric processing steps. During the weaving of textiles, the threads are exposed to considerable mechanical strain. Prior to weaving on mechanical looms, warp yarns are often coated with sizing starch or starch derivatives to increase their tensile strength and to prevent breaking. A PS4 variant can be applied during or after the weaving to remove these sizing starch or starch derivatives. After weaving, a PS4 variant can be used to remove the size coating before further processing the fabric to ensure a homogeneous and wash-proof result.

A PS4 variant can be used alone or with other desizing chemical reagents and/or desizing enzymes to desize fabrics, including cotton-containing fabrics, as detergent additives, e.g., in aqueous compositions. A PS4 variant also can be used in compositions and methods for producing a stonewashed look on indigo-dyed denim fabric and garments. For the manufacture of clothes, the fabric can be cut and sewn into clothes or garments, which are afterwards finished. In particular, for the manufacture of denim jeans, different enzymatic finishing methods have been developed. The finishing of denim garment normally is initiated with an enzymatic desizing step, during which garments are subjected to the action of amylolytic enzymes to provide softness to the fabric and make the cotton more accessible to the subsequent enzymatic finishing steps. A PS4 variant can be used in methods of finishing denim garments (e.g., a “bio-stoning process”), enzymatic desizing and providing softness to fabrics, and/or finishing process.

### 6. DP4 PRODUCTION FROM GRANULAR STARCH USING PS4

In another aspect a method of making a saccharide syrup, including adding a PS4 variant and an alpha-amylase to granular starch and hydrolyzing the granular starch to form the saccharide syrup is provided. In one embodiment the PS4 variant is added to the granular starch in a range from about 0.001% by weight to about 0.1% by weight based on dissolved solids. In another embodiment the PS4 variant is added to the granular starch in a range from about 0.0025% by weight to about 0.01% by weight based on dissolved solids. The granular starch can be obtained from starch from corns, cobs, wheat, barley, rye, milo, sago, cassava, tapioca, sorghum, rice, peas, bean, banana, or potatoes.

In a particular embodiment the granular starch is saccharified at about 60° C. to about 65° C. In another embodiment the granular starch is saccharified at about pH 5.0 to about pH 7.0. It is envisioned that the method can also include fermenting the saccharide syrup to produce ethanol.

In one embodiment the method includes a step of adding an enzyme having debranching activity to the granular starch. The enzyme having debranching activity can include but is not limited to an isoamylase, a pullulanase, an isopullulanase, a neopullulanase or any combination thereof. It is also envisioned that the method can optionally include a further step of adding a protease, a cellulase, a hemicellulase, a lipase, a cutinase, a pectate liase or any combination thereof to the granular starch.

In one embodiment the saccharide syrup includes at least about 40% by weight maltotetraose based on total saccharide content. Alternatively, the saccharide syrup includes at least about 45% by weight maltotetraose based on total saccharide content. In another embodiment the saccharide syrup includes at least about 50% by weight maltotetraose based on total saccharide content. In a further embodiment the saccharide syrup includes from about 45% by weight to about 60% by weight maltotetraose based on total saccharide content.

It is envisioned that the PS4 variant of the method can be immobilized.

### 7. IMO PRODUCTION USING PS4

In another aspect a method is provided for making IMO, including adding a) a PS4 variant, b) an alpha-amylase, and c) a transglucosidase to starch in the form of a starch liquefact or granular starch and saccharifying the starch to form IMO. Any of a number of transgucosidase enzymes (TG) can be use, for example, TRANSGLUCOSIDASE L-500® (Danisco US Inc., Genencor Division).

It is envisioned that the IMO can be formed at an IMO number of at least 30, at least 40 and/or at least 45. In one embodiment the starch is obtained from corns, cobs, wheat, barley, rye, milo, sago, cassava, tapioca, sorghum, rice, peas, bean, banana, or potatoes.

### EXAMPLES

Unless otherwise indicated, all percentages are expressed in weight percent. HPLC chromatography was employed to determine distribution of saccharide products.

### Example 1

### DP4 Production

Maltodextrin (340 g: DE 9.9%; moisture content 6.03%) was dissolved in tap water (660 g) to make a slurry at 32% DS. The pH of the slurry was adjusted to pH 5.5, pH 6.0, or pH 7.0, and 0.025 kg/MTDS SAS3 were added. Two 100 g aliquots were removed and placed into two 150 mL flasks maintained at 60° C. or 63° C. for 22 hr and 26 hr. Products of the reactions were analyzed by HPLC. Samples for HPLC analysis were prepared by dilution 0.25:10 with HPLC-grade water prior to filtration through a 0.45 micron filter. HPLC conditions: Phenomenex Rezex ROA-Organic Acid(H+) column; mobile phase: water; 16 min. runtime @ 60° C.; 20 μL injection; R1 detector.

The saccharides produced in the various reactions are shown in TABLE 1. DP4+ refers to oligosaccharides with a degree of polymerization of 4 or more (e.g., DP4, DP5, DP6, DP7, etc.). The percent DP4 decreased slightly with prolonged reaction time; however, DP4 yields were generally greater than 40% of the total saccharide content over the pH range tested. Furthermore, the enzyme appears to be relatively thermostable in the temperature range tested, since the DP4 yield was approximately the same at 60° C. and 63° C.

TABLE 1

| | Temp | SAS3 | Time | DP1 | DP2 | DP3 | DP4 | DP4+ | DP3 + DP4 | |
|---|---|---|---|---|---|---|---|---|---|---|
| pH | (° C.) | (kg/MTDS) | (hr) | (%) | (%) | (%) | (%) | (%) | (%) | DE |
| 5.5 | 60 | 0.025 | 22 | 3.31 | 7.465 | 11.887 | 44.107 | 33.231 | 55.994 | 31.46 |
| | | | 26 | 3.533 | 7.623 | 12.368 | 43.932 | 32.543 | 56.3 | 31.79 |
| 6.0 | 60 | 0.025 | 22 | 3.343 | 7.472 | 11.921 | 44.077 | 33.187 | 55.998 | 31.49 |
| | | | 26 | 3.568 | 7.644 | 12.455 | 43.872 | 32.461 | 56.327 | 31.84 |
| 7.0 | 60 | 0.025 | 22 | 3.162 | 7.723 | 12.485 | 42.842 | 33.789 | 55.327 | 31.42 |
| | | | 26 | 3.457 | 7.913 | 13.176 | 42.832 | 32.623 | 56.008 | 31.89 |
| 5.5 | 63 | 0.025 | 22 | 3.238 | 7.396 | 12.108 | 44.387 | 32.87 | 56.495 | 31.45 |
| | | | 26 | 3.59 | 7.607 | 12.593 | 44.224 | 31.986 | 56.817 | 31.91 |
| 6.0 | 63 | 0.025 | 22 | 2.989 | 7.091 | 11.534 | 43.96 | 34.527 | 55.494 | 30.97 |
| | | | 26 | 3.343 | 7.315 | 12.221 | 44.344 | 32.776 | 56.565 | 31.53 |
| 7.0 | 63 | 0.025 | 22 | 3.127 | 7.519 | 12.459 | 43.368 | 33.527 | 55.827 | 31.37 |
| | | | 26 | 3.226 | 7.51 | 13.022 | 43.227 | 33.015 | 56.249 | 31.55 |

### Example 2

### DP4 Production

Raw cornstarch (745 g; moisture content 14%) was dissolved in tap water (1,255 g) to make a slurry at 32% DS. An intermediate liquefact was produced by adding 0.4 kg/MTDS GC828, a blend of SPEZYME FRED and SPEZYME XTRA (Danisco US Inc, Genencor Division, Wuxi, China), to the slurry and holding the temperature at 95° C. for 45 min. This intermediate liquefact was separated into 100 g aliquots in 150 mL flasks maintained at 60° C. The pH was adjusted to 6.0 or 7.0 with 20% sulfuric acid. SAS3 was added at the concentrations indicated in TABLES 2 and 3, and additional liquefaction was performed for 15 hr, 19 hr, or 40 hr. HPLC analysis of the liquefact was used to determine the levels of various DPn sugars, using the procedure described in Example 1. Sugar profiles of the product liquefact syrups are shown in TABLES 2 and 3.

TABLE 2

| | Temp | | Time | DP1 | DP2 | DP3 | DP4 | DP4+ | DP3 + DP4 | |
|---|---|---|---|---|---|---|---|---|---|---|
| pH | (° C.) | SAS3 | (hr) | (%) | (%) | (%) | (%) | (%) | (%) | DE |
| 6.0 | 60 | 0.010% | 15 | 4.369 | 7.081 | 12.586 | 45.03 | 30.932 | 57.616 | 32.43 |
| | | | 19 | 5.071 | 7.478 | 13.586 | 43.957 | 29.907 | 57.543 | 33.26 |
| | | | 40 | 7.978 | 9.01 | 15.762 | 38.183 | 29.066 | 53.945 | 36.04 |
| 6.0 | 60 | 0.030% | 15 | 7.748 | 8.836 | 16.094 | 38.627 | 28.696 | 54.721 | 35.91 |
| | | | 19 | 9.375 | 9.721 | 17.445 | 35.575 | 27.884 | 53.02 | 37.52 |
| | | | 40 | 16.142 | 12.479 | 19.45 | 25.034 | 26.895 | 44.484 | 43.36 |
| 6.0 | 60 | 0.050% | 15 | 11.451 | 10.47 | 18.268 | 32.098 | 27.712 | 50.366 | 39.29 |
| | | | 19 | 13.858 | 11.522 | 19.445 | 28.395 | 26.781 | 47.84 | 41.50 |
| | | | 40 | 24.323 | 14.758 | 19.734 | 15.476 | 25.708 | 35.21 | 49.92 |
| 6.0 | 60 | 0.075% | 15 | 15.842 | 12.023 | 19.681 | 25.47 | 26.985 | 45.151 | 43.04 |
| | | | 19 | 19.155 | 13.32 | 20.374 | 20.946 | 26.205 | 41.32 | 45.89 |
| | | | 40 | 33.02 | 16.215 | 18.267 | 8.139 | 24.359 | 26.406 | 56.45 |
| 6.0 | 60 | 0.100% | 15 | 19.205 | 13.047 | 20.197 | 21.151 | 26.401 | 41.348 | 45.81 |
| | | | 19 | 23.146 | 14.274 | 20.208 | 16.647 | 25.725 | 36.855 | 49.00 |
| | | | 40 | 38.594 | 16.466 | 16.302 | 4.852 | 23.785 | 21.154 | 60.31 |

TABLE 3

| | Temp | | Time | DP1 | DP2 | DP3 | DP4 | DP4+ | DP3 + DP4 | |
|---|---|---|---|---|---|---|---|---|---|---|
| pH | (° C.) | SAS3 | (hr) | (%) | (%) | (%) | (%) | (%) | (%) | DE |
| 7.0 | 60 | 0.010% | 15 | 2.696 | 6.331 | 10.568 | 46.394 | 34.011 | 56.962 | 30.49 |
| | | | 19 | 2.977 | 6.533 | 11.254 | 46.187 | 33.049 | 57.441 | 30.92 |
| | | | 40 | 3.791 | 7.207 | 12.039 | 45.605 | 31.358 | 57.644 | 31.96 |
| 7.0 | 60 | 0.030% | 15 | 4.166 | 7.097 | 12.355 | 45.353 | 31.029 | 57.708 | 32.26 |
| | | | 19 | 4.43 | 7.502 | 13.131 | 44.643 | 30.295 | 57.774 | 32.72 |
| | | | 40 | 6.945 | 9.089 | 15.505 | 39.598 | 28.863 | 55.103 | 35.34 |
| 7.0 | 60 | 0.050% | 15 | 4.639 | 7.506 | 13.201 | 44.473 | 30.181 | 57.674 | 32.89 |
| | | | 19 | 5.336 | 7.989 | 14.042 | 42.913 | 29.72 | 56.955 | 33.65 |
| | | | 40 | 8.639 | 10.003 | 16.756 | 36.219 | 28.383 | 52.975 | 36.96 |
| 7.0 | 60 | 0.075% | 15 | 5.635 | 8.136 | 14.37 | 42.261 | 29.598 | 56.631 | 33.95 |
| | | | 19 | 6.678 | 8.774 | 15.494 | 40.233 | 28.822 | 55.727 | 35.06 |
| | | | 40 | 11.406 | 11.321 | 18.514 | 31.284 | 27.475 | 49.798 | 39.55 |
| 7.0 | 60 | 0.100% | 15 | 6.544 | 8.637 | 15.337 | 40.315 | 29.167 | 55.652 | 34.88 |
| | | | 19 | 7.824 | 9.429 | 16.54 | 37.774 | 28.434 | 54.314 | 36.20 |
| | | | 40 | 13.761 | 12.368 | 19.347 | 27.384 | 27.14 | 46.731 | 41.62 |

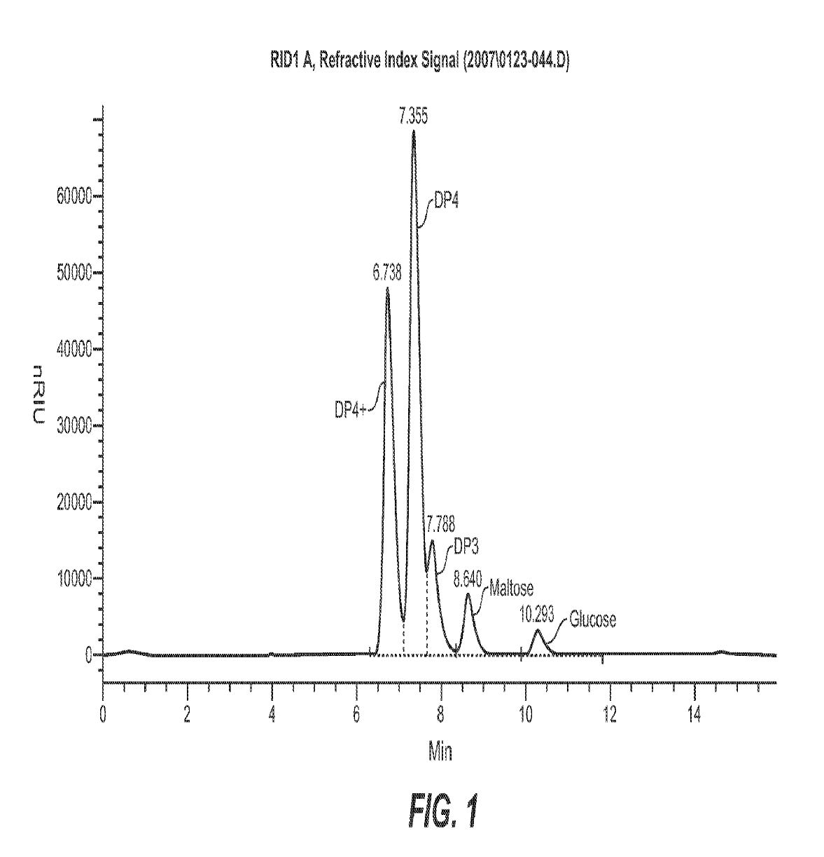

As shown in TABLES 2 and 3, DP4 yield decreased as reaction time was prolonged and at higher enzyme dosages. At ph 6.0, the DP4 yield decreased substantially with prolonged reaction time. Although DP4 yield also decreased with prolonged reaction time at pH 7.0, DP4 yields were more stable, and several samples provided a DP4 yield greater than 45% of the total saccharide content. For example, at an SAS3 dose of 0.01%, DP4 levels rose above 45% at both pH 6.0 and 7.0 after a 15 hr reaction. In most of the samples at pH 7.0 and below, the DE reached a desirable range of 30-40. FIG. 1 depicts an exemplary chromatogram of liquefied corn starch saccharified according to the above procedure at 0.01% SAS3, pH 7.0 for 19 hr.

### Example 3

### DP4 Production

Raw cornstarch was liquefied as in Example 2 to provide an intermediate liquefact containing 0.577% DP1, 3.145% DP2, 6.489% DP3, and 89.789% DP4+ and having a DE of 21.13. This intermediate liquefact was separated as 100 g aliquots in six 150 mL flasks and tested in duplicate at three enzyme doses at pH 7.0. After dosing with an amount of SAS3 shown in TABLE 4, each flask was shaken and heated in a 60° C. water bath for 16 hr, 19 hr and 40 hr. HPLC analysis of the liquefact was used to determine comparative levels of DPn sugars as in Example 1. Sugar profiles of the product liquefact syrups are shown in TABLE 4.

TABLE 4

| | Temp | | Time | DP1 | DP2 | DP3 | DP4 | DP4+ | DP3 + DP4 | |
|---|---|---|---|---|---|---|---|---|---|---|
| pH | (° C.) | SAS3 | (hr) | (%) | (%) | (%) | (%) | (%) | (%) | DE |
| 7.0 | 60 | 0.1 kg/ | 16 | 7.275 | 11.455 | 18.908 | 36.615 | 25.747 | 55.523 | 36.93 |
| | | MTDS | 20 | 8.41 | 12.259 | 20.014 | 34.598 | 24.719 | 54.612 | 38.19 |
| | | | 24 | 9.35 | 12.61 | 20.448 | 33.163 | 24.429 | 53.611 | 39.02 |
| 7.0 | 60 | 0.1 kg/ | 16 | 7.481 | 11.464 | 19.031 | 36.391 | 25.633 | 55.422 | 37.11 |
| | | MTDS | 20 | 8.747 | 12.38 | 20.053 | 34.244 | 24.576 | 54.297 | 38.48 |
| | | | 24 | 9.638 | 12.698 | 20.632 | 32.925 | 24.107 | 53.557 | 39.30 |
| 7.0 | 60 | 0.075 kg/ | 16 | 6.248 | 11.303 | 18.444 | 37.874 | 26.13 | 56.318 | 36.08 |
| | | MTDS | 20 | 7.075 | 11.895 | 19.548 | 36.297 | 25.185 | 55.845 | 37.05 |
| | | | 24 | 7.726 | 12.261 | 19.967 | 35.222 | 24.824 | 55.189 | 37.69 |
| 7.0 | 60 | 0.075 kg/ | 16 | 6.266 | 11.234 | 18.437 | 37.911 | 26.152 | 56.348 | 36.07 |
| | | MTDS | 20 | 7.118 | 11.907 | 19.346 | 36.409 | 25.22 | 55.755 | 37.06 |
| | | | 24 | 7.832 | 12.255 | 20.05 | 35.135 | 24.71 | 55.185 | 37.78 |
| 7.0 | 60 | 0.05 kg/ | 16 | 5.402 | 11.358 | 18.253 | 38.318 | 26.669 | 56.571 | 35.42 |
| | | MTDS | 20 | 6.005 | 11.871 | 18.985 | 37.47 | 25.669 | 56.455 | 36.18 |
| | | | 24 | 6.53 | 12.281 | 19.769 | 36.362 | 25.057 | 56.131 | 36.81 |
| 7.0 | 60 | 0.05 kg/ | 16 | 5.554 | 11.294 | 18.254 | 38.41 | 26.488 | 56.664 | 35.53 |
| | | MTDS | 20 | 6.185 | 11.892 | 19.191 | 37.188 | 25.544 | 56.379 | 36.34 |
| | | | 24 | 6.753 | 12.242 | 19.846 | 36.249 | 24.91 | 56.095 | 36.98 |

As shown in TABLE 4, DP4 yield remained steady at concentrations of SAS3 as low as 0.05 kg/MTDS. This level of enzyme provided useful DP4 levels in a range of 35-39% by weight based on total saccharide, while DE was in a desirable range of 35-40. Also, the yield of DP3 was somewhat higher compared to previous examples under these conditions.

### Example 4

### Expression of PS4 Variant SAS3

SAS3 was expressed in Bacillus licheniformis using an IPTG-inducible pET expression vector, according to known methods. After purification, filtration, and concentration, inclusion bodies containing the enzyme were isolated, and the enzyme was renatured in 50 mM sodium citrate (pH 6.5) at 60° C. A stock solution was prepared at an enzyme concentration of 3 mg/mL.

### Example 5

### Treatment of Maltodextrin with SAS3

MALTRIN® M040 (water content 6.38% by weight) was dissolved in tap water to make a slurry at 32% DS. The pH was adjusted to 6.5 using 0.1 M sodium carbonate. The slurry was added in aliquots of 2, 3, or 4 grams into glass test tubes. The sample tubes were capped with a plastic cover, stirred, and placed in a 60° C. water bath. Aliquots (0.02 mL) were removed at the measured time intervals, dissolved in 0.01 N sulfuric acid, and analyzed by HPLC.

HPLC analysis was performed using an Agilent 1200 Series (Agilent Technologies, Palo Alto, Calif.) equipped with an Aminex HPX-87H column (300×7.8 mm) with guard at 60° C.; eluent 0.01 N sulfuric acid; flow rate 0.6 mL/min.; refractive index (RI) detector at 55° C.; runtime 15 or 24 min. A volume of 0.02 mL of sample as injected (2% in 0.01 N sulfuric acid of incubation mixture). Commercial standards of glucose (DP1), maltose (DP2), maltotriose (DP3), maltotetraose (DP4), maltopentaose (DP5) and maltohexaose (DP6) at four different concentrations were used to calibrate the R1 response of the samples. Processing of the signals was preformed using ChemStation for LC 3D software (Agilent Technologies).

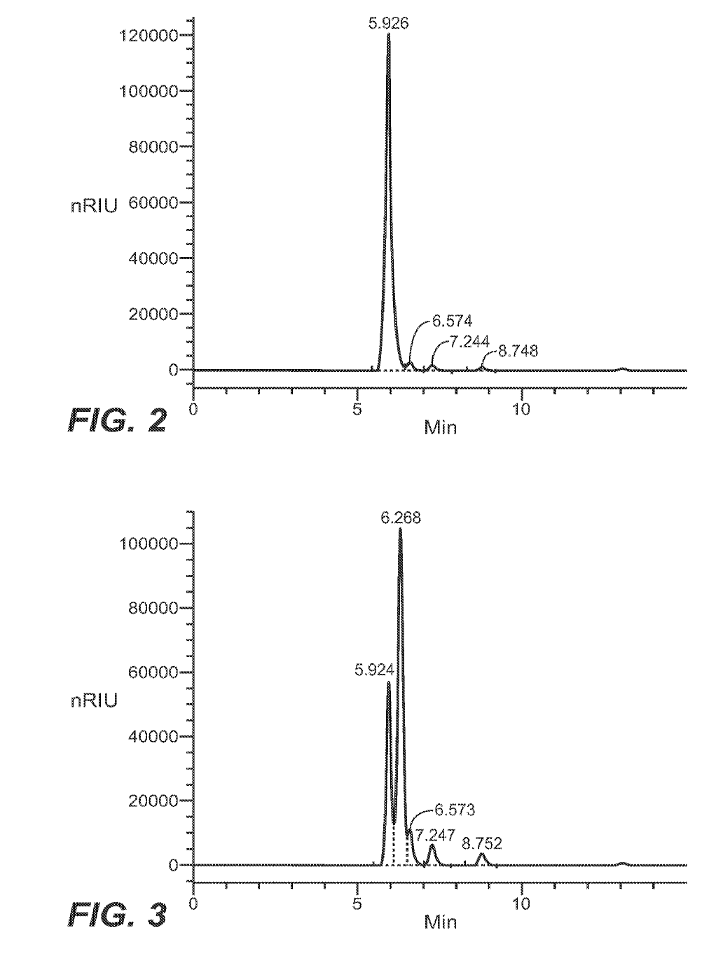

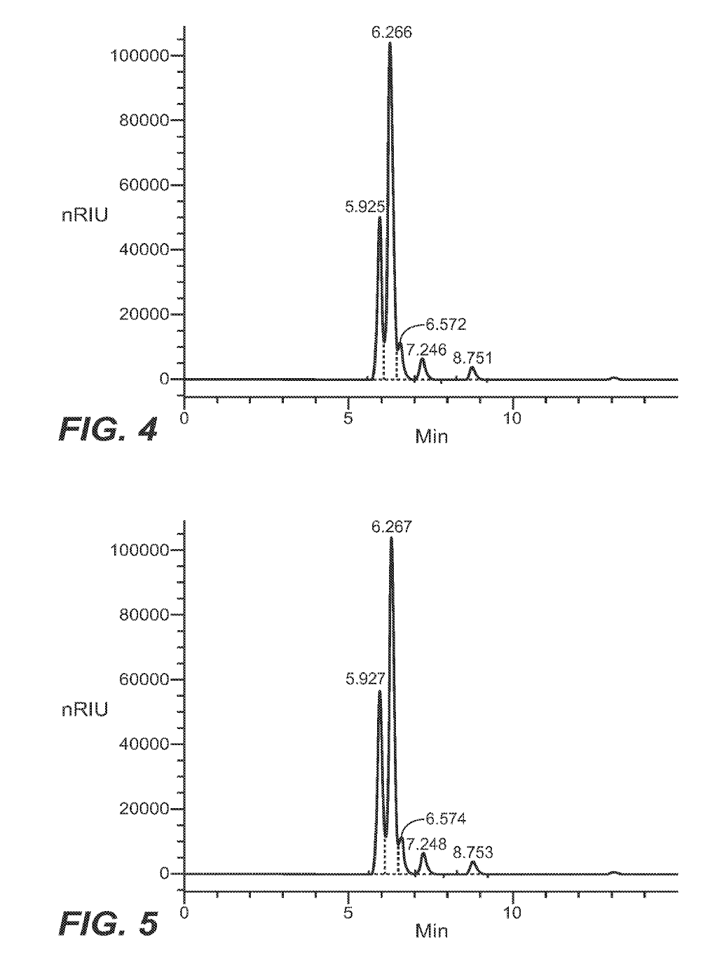

Maltodextrin slurry (2.0 g per sample) was inoculated with SAS3 at concentrations of 0 (control), 0.025, 0.05, and 0.1 kg/MTDS. As shown in FIG. 2, the control sample showed no breakdown of the substrate. In the presence of 0.025 kg/MTDS SAS3, however, significant breakdown of the substrate was apparent, as shown in FIG. 3. Likewise, FIG. 4 depicts the substrate breakdown using 0.05 kg/MTDS SAS3, and FIG. 5 depicts the substrate breakdown using 0.1 kg/MTDS SAS3. In contrast to other amylases that produce glucose or maltose, SAS3 preferentially produces maltotetraose (DP4), as shown in FIG. 3-5. The yields of maltotetraose exceed 55% by weight based on total saccharide under these conditions.

### Example 6

### Time Progress Curve for Production of Maltotetraose

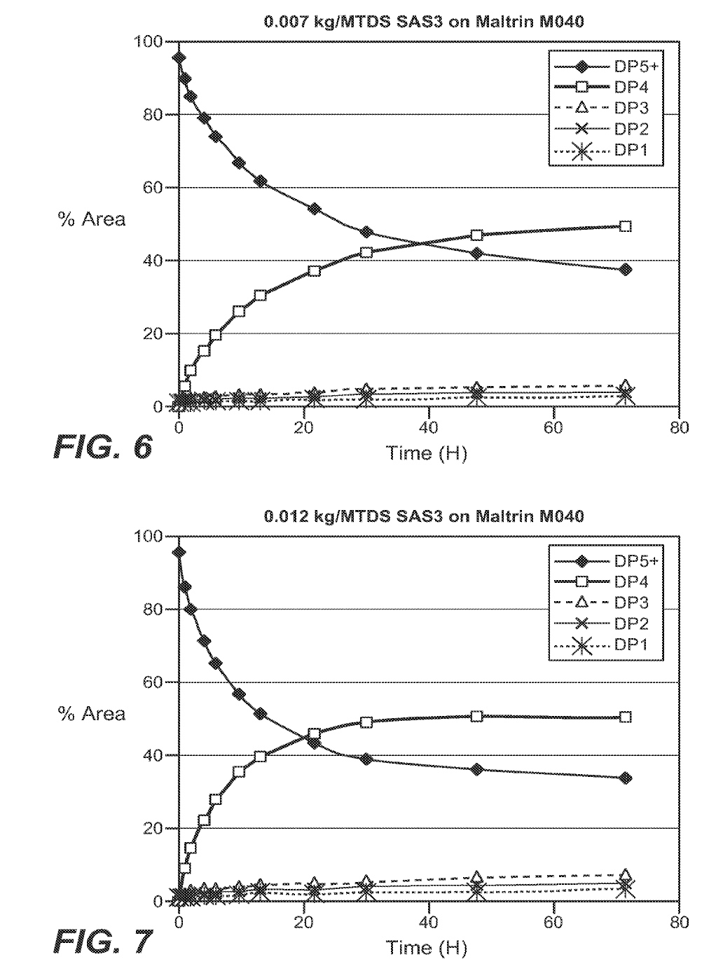

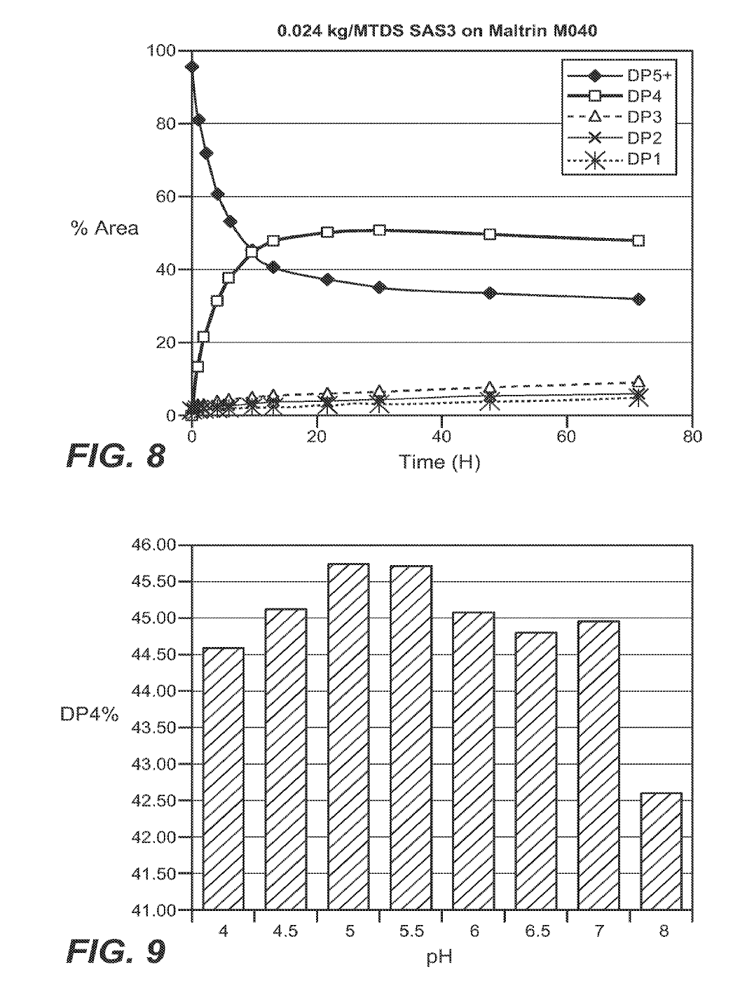

The maltodextrin slurry of Example 5 was adjusted to pH 5.5 (4 g per sample) and inoculated with SAS3 at concentrations of 0.007, 0.012, and 0.024 kg/MTDS. The samples were heated in a 60° C. water bath and monitored by HPLC over a period of 72 hours. FIG. 6-8 show the appearance of reaction products expressed as DP1, DP2, DP3, DP4, and DP5+ over time, using 0.007, 0.012, and 0.024 kg/MTDS SAS3, respectively.

In comparison to Example 5, the maximum amount of maltotetraose (DP4) produced was about 50%, although the maximum amount of DP4 was produced at different times using the different SAS3 concentrations. SAS3 remains active after 30 hours, as evidenced by continued production of DP4 at lower enzyme concentrations.

### Example 7

### Maltotetraose Content Assay by HPLC

In the following examples, the content of maltotetraose in a syrup treated with SAS3 was assayed by an HPLC system consisting of a resin column (phenomenex Rezex-ROA-H⁺) and Reference detector (RI, Agilent Co, USA). Samples (20 μL, 1% w/v) were injected, and the column was eluted at 0.6 mL/min with a linear gradient of 0.05N sulfuric acid. The column and detector were kept at 60° C. and 35° C., respectively.

### Example 8

### Optimal pH of SAS3

In this example, experiments were conducted to measure the optimal operational pH for SAS3 to produce the highest content maltotetraose syrup. Maltodextrin (213 g, DE 9.9, pH 5.3, 0.1% ash) and tap water (787 g) were mixed to prepare DS 20% slurry, according to the maltodextrin moisture 6.03%. The slurry was aliquoted in 100 gram quantities into 150 ml flasks and the pH was adjusted to pH 4.0 to pH 8.0 with 20% (w/v) sulfuric acid. SAS3 was diluted 1:100 with RO water prior to dosing, and 0.025 kg/MTDS SAS3 were added to every flask. The reactions were run at 60° C., and samples were taken at 17 hr, 22 hr and 48 hr. As shown in FIG. 9, the optimal pH range for SAS3 production of maltotetraose was from about 5.0 to 5.5.

### Example 9

### Optimal Temperature of SAS3

In this example, experiments were conducted to measure optimal operational temperature for SAS 3 to produce the highest content maltotetraose syrup. Maltodextrin (213 g, DE 9.9, pH 5.3, 0.1% ash) and tap water (787 g) were mixed to prepare DS 20% slurry, according to the maltodextrin moisture 6.03%. The slurry was aliquoted in 100 gram quantities into 150 ml flasks and adjusted pH to 5.0 with 20% (w/v) sulfuric acid. SAS3 was diluted 1:100 with RO water prior to dosing. 0.025 kg/MTDS SAS3 was added to every flask. The reactions were run at 50° C. to 70° C., and samples were taken at 17 hr and 22 hr. As shown in FIG. 10, the optimal temperature range for SAS3 production of maltotetraose was about 60° C. to 65° C.

### Example 10

### Optimal Dosage of SAS3

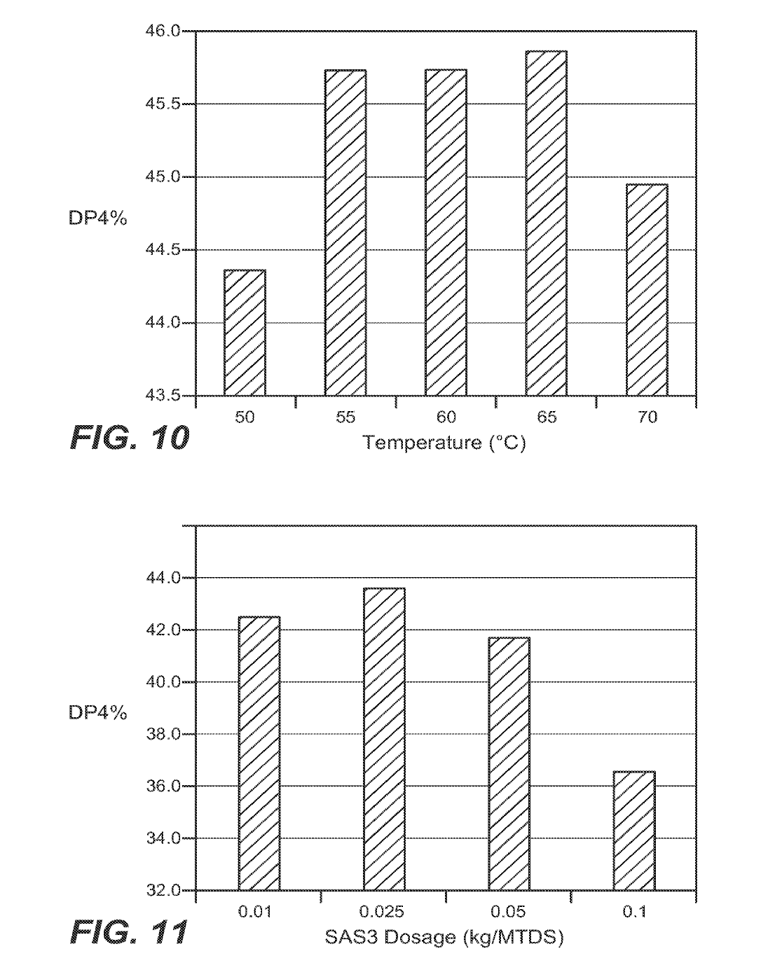

In this example, experiments were conducted to measure optimal SAS 3 dosage for producing the highest content maltotetraose syrup. Maltodextrin (213 g, DE 9.9, pH 5.3, 0.1% ash) and tap water (787 g) were mixed to prepare DS 20% slurry, according to the maltodextrin moisture 6.03%. The slurry was aliquoted in 100 gram quantities into 150 ml flasks and adjusted pH to 5.0 with 20% (w/v) sulfuric acid. SAS3 was diluted 1:100 with RO water prior to dosing. SAS3 was added at 0.01, 0.025, 0.05, and 0.1 kg/MTDS. The reactions were run at 60° C., and samples were taken at 17 hr and 22 hr. As shown in FIG. 11, the optimal dosage for SAS3 production of maltotetraose under these conditions was 0.025 kg/MDTS.

### Example 11

### Addition of Pullulanase to Increase Maltotetraose Yield

In this example, experiments were conducted demonstrate that pullulanase could help SAS 3 to increase maltotetraose yield and to test for pullulanase dosage. Maltodextrin (213 g, DE 9.9, pH 5.3, 0.1% ash) and tap water (787 g) were mixed to prepare DS 20% slurry, according to the maltodextrin moisture 6.03%. The slurry was aliquoted in 100 gram quantities into 150 ml flasks and adjusted pH to 5.0 with 20% (w/v) sulfuric acid. SAS3 and pullulanase (Optimax L-1000, Danisco US Inc, Genencor Division) were diluted 1:100 with RO water prior to dosing. SAS3 was added at 0.025 kg/MDTS, and pullulanase was added to 0.1, 0.25, 0.5, 1. or 1.5 kg/MTDS. The reactions were run at 65° C., and samples were taken at 17 hr and 22 hr. As shown in FIG. 12, maltotetraose yield increased as the dosage of pullulanase increased.

### Example 12

### Initial Dry Solids Content Effect on Maltotetraose Yield

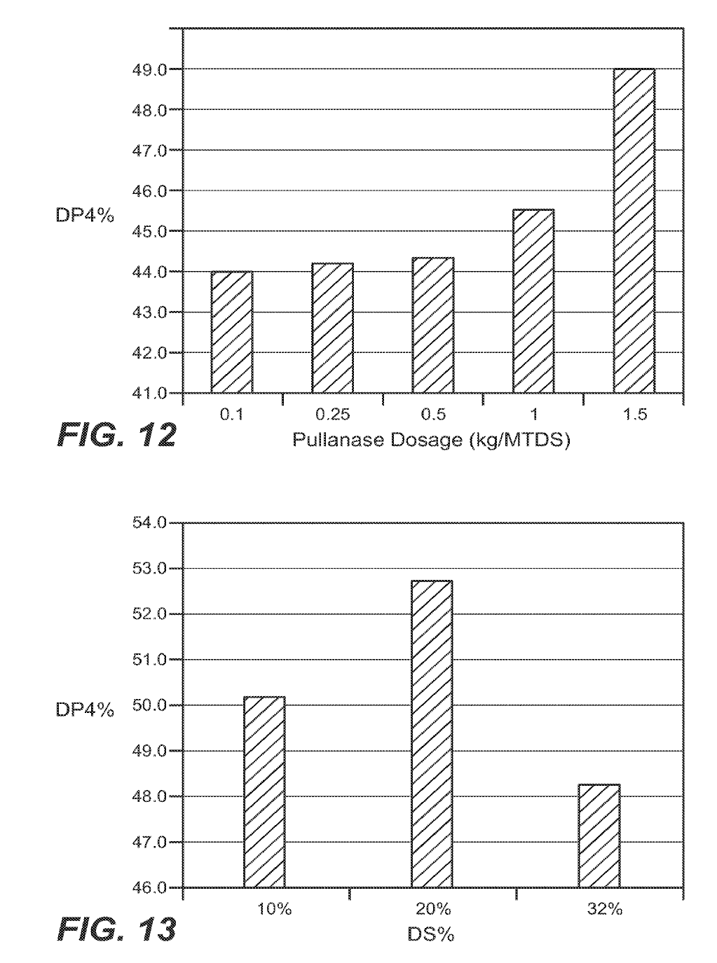

Maltodextrin (DE 9.9, pH 5.3, 0.1% ash) 10.6 g, 21.3 g, and 34 g, and tap water 89.4 g, 78.7 g, and 66 g, were mixed to prepare DS 10%, 20%, and 32% slurries, respectively, according to the maltodextrin moisture 6.03%. The slurries were adjusted pH to 5.0 with 20% (w/v) sulfuric acid. SAS3 and pullulanase (Optimax L-1000, Danisco US Inc, Genencor Division) were diluted 1:100 with RO water prior to dosing. 0.01 kg/MDTS SAS3 and 1 kg/MDTS pullulanase were added. The reactions were run at 65° C., and samples were taken at 17 hr and 22 hr. As shown in FIG. 13, an substrate concentration of 20% DS provides the highest maltotetraose yield.

### Example 13

### DP4 Production from Granular Starch Using SPEZYME® ALPHA+SAS3

### Materials and Methods

| | | |
|---|---|---|
| Enzymes | SPEZYME ® ALPHA | SAS3 |

| | | | | |
|---|---|---|---|---|
| Activity | 14478 | AAU/g | 50 | BMK/g |
| Dose | 2 | AAU/gds | 0.03 | BMK/gds |

100 g of 32% ds starch slurry was pH adjusted to 5.3 and then dosed with 2 AAU/gds of SPEZYME® ALPHA and 0.03 BMK/gds of SAS3 at 60 degrees C. for DP4 production. Reactions were carried out for up to 15 hours before removing samples. Samples were centrifuged to produce a supernatant that was treated in boiling water to deactivate enzymes. Percent solubility was calculated by ratio of brix of each sample to that of a completely solubilized sample.

The enzyme-deactivated samples were diluted by taking 0.5 ml sample and combining it with 4.5 ml of RO water. The mixture was then filtered through 0.45 μm Whatman filters and put into vials for HPLC analysis. The HPLC analysis was conducted using a REZEX™ ROA-organic Acid H+ column with a guard column.

### Results and Discussion

TABLE 7

| Dosage of | | | | | | % |
|---|---|---|---|---|---|---|
| SPEZYME ® ALPHA + SAS3 | % DP1 | % DP2 | % DP3 | % DP4 | % HS | Solubility |
| (2 AAU + 0.03 BMK)/g ds | 4.26 | 14.92 | 19.82 | 40.65 | 20.36 | 54.3 |

As shown in Table 7, SAS3 produced DP4 as a major product by 40.65% with 54.3% solubility in 15 hours. SAS3 typically produces DP4 up to 45˜50% with conventional substrate, i.e., liquefied starch, but in this case, DP4 was lower with higher DP2 and DP3. The higher DP2 and DP3 may be due to alpha-amylase activity in the substrate as it has been reported that residual alpha-amylase activity results in increased DP2 and DP3 during saccharification. Still, this result indicates that granular starch is a suitable SAS3 substrate for DP4 production in the presence of alpha-amylase.

### Example 14

### IMO Production from SPEZYME® FRED Liquefied Starch Using SAS3+TRANSGLUCOSIDASE L-500® (Danisco US Inc., Genencor Division)

### Materials and Methods

| | | |
|---|---|---|
| | | TRANSGLUCOSIDASE |
| Enzymes | SAS3 | L-500 ® |

| | | | | |
|---|---|---|---|---|
| Activity | 50 | BMK/g | 500 | TGU/g |
| Dose | 0.03 | BMK/gds | 1.4 | KG/MTds |

SPEZYME® FRED starch liquefact (˜9.1DE) was pH adjusted to 5.35 with NaOH after which each 100 g of liquefact was incubated at 60 degrees C. for saccharification by dosing SAS3+TRANSGLUCOSIDASE L-500®. Reactions were carried out for up to 48 hours with periodical samplings. Samples were treated in boiling water to deactivate enzymes.

The enzyme-deactivated samples were diluted by taking 0.5 ml sample and combining it with 4.5 ml of RO water. Samples were then filtered through 0.45 μm Whatman filters and put into vials for HPLC analysis. The HPLC analysis was conducted using a REZEX™ ROA-organic Acid H+ column followed by Shodex RSpak DC-613 to identify IMO having α-1,6 bond.

### Results and Discussion

TABLE 8

| Dosage of SAS3 + | | | | | | | | |
|---|---|---|---|---|---|---|---|---|
| TRANSGLUCOSIDASE | % | % | % | % | % | % | % | IMO |
| L-500 ® | Glucose | Maltose | Isomaltose | Maltotriose | Panose | Maltotetraose | Isomaltotriose | No. |
| (0.03 BMK + 1.3 mg)/g ds | 19.12 | 13.79 | 7.36 | 8.93 | 19.47 | 11.3 | 20.1 | 46.89 |

Table 8 shows that the SAS3+TRANSGLUCOSIDASE L-500® combination successfully produced significant amount of isomalto-oligosaccharides such as isomaltose, panose and isomaltotriose, giving 46.89 as IMO number, which is calculated based on the sum of % amount of all of isomalto-oligosaccharides. This result indicates that more economical substrate such as liquefied starch can be used for IMO production instead of relatively costly high maltose syrup in conventional processes.

It will be apparent to those skilled in the art that various modifications and variation can be made to the compositions and methods of using the same without departing from the spirit or scope of the intended use. Thus, it is the modifications and variations provided they come within the scope of the appended claims and their equivalents. All references cited above are herein incorporated by reference in their entirety for all purposes.

TABLE 5

| | | | | | | | | | |
|---|---|---|---|---|---|---|---|---|---|
| A3S | G70D | V113I | G134C | G158T | A179N | G223P | W232P | G303L | R316P |
| A3T | G70K | N116D | R137C | G158F | A179R | G223I | W232Q | G303E | R316K |
| P7S | G70E | N119S | N138D | G158P | A179E | G223L | W232R | G303D | W323M |
| A8N | G70S | N119G | N138E | G158I | A179T | G223V | W232S | Q305E | T324L |
| G9A | G70Q | N119Y | N138S | G158A | R182S | G223C | W232Y | Q305T | T324M |
| H13R | G70A | N119E | C140R | G158V | R182H | G223T | W232T | Q305L | T324A |
| N26E | G70V | G121W | C140A | G158L | R182M | G223S | R233H | H307D | S325G |
| N26D | G70L | G121A | A141S | G158Q | R182D | G223Y | N234R | H307L | S334R |
| P32S | G70P | G121F | A141P | G158C | R182G | G223W | A236E | H307R | S334Q |
| N33Y | K71R | G121L | D142N | E160D | S183G | G223Q | S237G | H307K | S334H |
| D34N | K71M | G121T | D142G | S161V | G184Q | G223N | S237D | H307G | S334A |
| I38M | S72E | G121S | D142E | S161A | G188A | G223D | W238Q | H307P | S334M |
| I46F | S72K | G121E | P143T | S161T | G188H | G223H | W238G | H307I | S334L |
| D49V | S72N | G121K | G144E | S161K | G188T | G223K | W238K | H307S | S334P |
| D62N | S72T | G121R | N145D | S161P | G188S | G223R | W238R | H307M | H335M |
| F63L | G73M | G121H | N145S | S161G | F192Y | G223M | W238P | H307Q | W339E |
| F63A | G73S | G121M | Y146G | S161R | F192F | G223A | W238E | H307V | W339A |
| F63D | G73T | G121V | Y146E | S161H | F192M | G223E | Q239L | H307W | Y341E |
| F63E | G73N | G121P | Y146D | L163M | V195D | G223F | V253G | H307Y | Y341C |
| F63V | G73L | G121I | N148S | N164R | R196P | S225G | D255V | H307C | D343E |
| S64T | G73E | G121D | N148K | G166N | R196Q | S225E | A257V | H307F | R353T |
| S64N | G73D | Y122W | D149V | P168L | R196T | S225V | E260R | H307E | R358A |
| T67V | G74S | Y122A | D149L | Q169R | R196K | E226W | E260K | W308C | R358T |
| T67K | G75C | Y122Q | D149H | Q169K | R196Y | E226C | N264D | W308T | R358L |
| T67Q | G75S | Y122E | C150A | Q169V | R196S | E226D | V267I | W308K | R358V |
| T67H | G75R | P123S | D151W | Q169G | R196G | E226G | D269V | W308N | R358Q |
| T67R | G75Y | D124S | D151A | Q169E | R196A | Y227G | D269S | W308R | R358E |
| T67G | G75F | K125E | D151V | Q169N | R196V | Y227T | D269N | W308S | R358N |
| T67N | G75W | K125G | G153D | Q169D | Y198W | Y227D | K271L | W308G | R358G |
| D68C | G75E | K125A | S334K | I170M | Y198F | Y227K | K271Q | W308Q | S367R |
| D68E | E76V | K125W | S334T | I170E | A199P | Y227C | K271A | W308A | S367Q |
| G69M | G100A | K125D | G153A | I170L | P200G | S229N | H272Q | A309T | S379G |
| G69I | G100S | K125Q | D154G | I170K | P200A | S229P | G276R | A309E | D390E |
| G69H | G104R | K125P | D154E | I170N | R202K | W232F | W282S | A309M | S399P |
| G69E | G104N | E126N | D154Y | L178N | S208T | W232G | V285A | A309V | S420G |
| G69A | G106K | E126D | F156Y | L178W | S213N | W232H | V290I | A309I | D422N |
| G69R | V107M | N128E | I157L | L178Q | L220A | W232I | T295C | A309P | D422Q |
| G69P | L110F | P130S | I157V | L178F | L220T | W232K | Y297H | D312E | D422P |
| G69T | D112E | A131T | I157M | A179P | K222Y | W232L | G300E | R316Q | G424S |
| G69K | | G134R | G158S | A179S | K222M | W232N | N302K | R316S | G424D |

TABLE 6

| Backbone | Mutations |
|---|---|
| SAS3 | N33Y, D34N, G70D, G121F, G134R, A141P, Y146G, |
| | I157L, S161A, L178F, A179T, G223E, S229P, H307K, |
| | A309P, S334P |

What is claimed is:

1. A Pseudomonas saccharophila amylase (PS4) variant of a wild-type PS4 having the amino acid sequence of SEQ ID NO: 2, comprising:
(i) a G223E amino acid substitution, and
(ii) up to 24 additional amino acid deletions, additions, insertions, or substitutions compared to the amino acid sequence of SEQ ID NO: 2; or
(iii) at least 70% sequence identity to the amino acid sequence of SEQ ID NO: 2, wherein the PS4 variant has α-amylase activity.

2. The PS4 variant of claim 1, further comprising a methionine at the polypeptide sequence amino terminus.

3. The PS4 variant of claim 1, wherein the PS4 variant comprises a polypeptide sequence with up to 15 additional amino acid substitutions compared to the amino acid sequence of SEQ ID NO: 2.

4. The PS4 variant of claim 3, comprising one or more of the following amino acid substitutions: N33Y, D34N, G70D, G121F, G134R, A141P, Y146G, I157L, S161A, L178F, A179T, S229P, H307K, A309P, or S334P.

5. The PS4 variant of claim 4, wherein the PS4 variant comprises the amino acid sequence of SEQ ID NO: 3.

6. The PS4 variant of claim 1, wherein the PS4 variant has at least 80% sequence identity to the amino acid sequence of SEQ ID NO: 2.

7. The PS4 variant of claim 6, wherein the PS4 variant has at least 90% sequence identity to the amino acid sequence of SEQ ID NO: 2.

8. The PS4 variant of claim 7, wherein the PS4 variant has at least 95% sequence identity to the amino acid sequence of SEQ ID NO: 2.

9. The PS4 variant of claim 1, wherein the PS4 variant is purified.

10. The PS4 variant of claim 1, wherein the PS4 variant has an altered thermostability compared to the wild-type PS4.

11. The PS4 variant of claim 10, wherein the PS4 variant is more thermostable than the wild-type PS4.

12. The PS4 variant of claim 1, wherein the PS4 variant is more stable than the wild-type PS4 at a pH of about 5.0 to about 7.0.

13. The PS4 variant of claim 1, wherein the PS4 variant has more exo-α-amylase activity than the wild-type PS4.

14. The PS4 variant of claim 1, wherein the PS4 variant has more endo-α-amylase activity than the wild-type PS4.

15. A polynucleotide that encodes a PS4 variant according to claim 1.

16. An expression vector comprising the polynucleotide of claim 15.

17. A host cell comprising the expression vector of claim 16.

18. A host cell that expresses the polynucleotide of claim 15.

19. A starch processing composition comprising the PS4 variant of claim 1.

20. A method of making a saccharide syrup, comprising adding a PS4 variant of claim 1 to a starch liquefact and saccharifying the starch liquefact to form the saccharide syrup.

21. The method of claim 20, wherein the PS4 variant is added to the starch liquefact in a range from about 0.001% by weight to about 0.1% by weight based on dissolved solids.

22. The method of claim 21, wherein the PS4 variant is added to the starch liquefact in a range from about 0.0025% by weight to about 0.01% by weight based on dissolved solids.

23. The method of claim 20, wherein the starch liquefact is obtained from starch from corns, cobs, wheat, barley, rye, milo, sago, cassava, tapioca, sorghum, rice, peas, bean, banana, or potatoes.

24. The method of claim 20, wherein the starch liquefact is saccharified at about 60° C. to about 65° C.

25. The method of claim 20, wherein the starch liquefact is saccharified at about pH 5.0 to about pH 7.0.

26. The method of claim 20, further comprising fermenting the saccharide syrup to produce ethanol.

27. The method of claim 20, further comprising a step of adding an enzyme having debranching activity to the starch liquefact.

28. The method of claim 27, wherein the enzyme having debranching activity is, an isoamylase, a pullulanase, an isopullulanase, a neopullulanase or any combination thereof.

29. The method of claim 27, optionally comprising a further step of adding a protease, a cellulase, a hemicellulase, a lipase, a cutinase, a pectate liase or any combination thereof to the starch liquefact.

30. The method of claim 20, wherein the saccharide syrup comprises at least about 40% by weight maltotetraose based on total saccharide content.

31. The method of claim 30, wherein the saccharide syrup comprises at least about 45% by weight maltotetraose based on total saccharide content.

32. The method of claim 31, wherein the saccharide syrup comprises at least about 50% by weight maltotetraose based on total saccharide content.

33. The method of claim 32, wherein the saccharide syrup comprises from about 45% by weight to about 60% by weight maltotetraose based on total saccharide content.

34. The method of claim 20, wherein the PS4 variant is immobilized.

35. A method of making a saccharide syrup, comprising adding a PS4 variant of claim 1 and an alpha-amylase to granular starch and hydrolyzing the granular starch to form the saccharide syrup.

36. The method of claim 35, wherein the PS4 variant is added to the granular starch in a range from about 0.001% by weight to about 0.1% by weight based on dissolved solids.

37. The method of claim 35, wherein the PS4 variant is added to the granular starch in a range from about 0.0025% by weight to about 0.01% by weight based on dissolved solids.

38. The method of claim 35, wherein the granular starch is obtained from starch from corns, cobs, wheat, barley, rye, milo, sago, cassava, tapioca, sorghum, rice, peas, bean, banana, or potatoes.

39. The method of claim 35, wherein the granular starch is saccharified at about 60° C. to about 65° C.

40. The method of claim 35, wherein the granular starch is saccharified at about pH 5.0 to about pH 7.0.

41. The method of claim 35, further comprising fermenting the saccharide syrup to produce ethanol.

42. The method of claim 35, further comprising a step of adding an enzyme having debranching activity to the granular starch.

43. The method of claim 42, wherein the enzyme having debranching activity is, an isoamylase, a pullulanase, an isopullulanase, a neopullulanase or any combination thereof.

44. The method of claim 42, optionally comprising a further step of adding a protease, a cellulase, a hemicellulase, a lipase, a cutinase, a pectate liase or any combination thereof to the granular starch.

45. The method of claim 35, wherein the saccharide syrup comprises at least about 40% by weight maltotetraose based on total saccharide content.

46. The method of claim 45, wherein the saccharide syrup comprises at least about 45% by weight maltotetraose based on total saccharide content.

47. The method of claim 46, wherein the saccharide syrup comprises at least about 50% by weight maltotetraose based on total saccharide content.

48. The method of claim 47, wherein the saccharide syrup comprises from about 45% by weight to about 60% by weight maltotetraose based on total saccharide content.

49. The method of claim 35, wherein the PS4 variant is immobilized.

50. A method of making IMO, comprising adding a) a PS4 variant of claim 1, b) an alpha-amylase, and c) a transglucosidase to starch in the form of a starch liquefact or granular starch and saccharifying the starch to form IMO.

54. The method of claim 50, wherein the starch is obtained from corns, cobs, wheat, barley, rye, milo, sago, cassava, tapioca, sorghum, rice, peas, bean, banana, or potatoes.

55. A textile desizing composition comprising a PS4 variant according to claim 1 in an aqueous solution, and optionally with another enzyme.

56. A method of desizing a textile, comprising contacting the desizing composition of claim 37 with a textile for a time sufficient to desize the textile.


SEQUENCE LISTING (4 sequences)

SEQ ID NO: 1 - protein, 551 aa, Pseudomonas saccharophila
  misc_feature: Protein sequence of G4-amylase from Pseudomonas saccharophila (including signal sequence)
MSHILRAAVLAAVLLPFPALADQAGKSPAGVRYHGGDEIILQGFHWNVVREAPNDWYNILRQQASTIAADGFSAIWMPVPWRDFSSWTDGGKSGGGEGYFWHDFNKNGRYGSDAQLRQAAGALGGAGVKVLYDVVPNHMNRGYPDKEINLPAGQGFWRNDCADPGNYPNDCDDGDRFIGGESDLNTGHPQIYGMFRDELANLRSGYGAGGFRFDFVRGYAPERVDSWMSDSADSSFCVGELWKGPSEYPSWDWRNTASWQQIIKDWSDRAKCPVFDFALKERMQNGSVADWKHGLNGNPDPRWREVAVTFVDNHDTGYSPGQNGGQHHWALQDGLIRQAYAYILTSPGTPVVYWSHMYDWGYGDFIRQLIQVRRTAGVRADSAISFHSGYSGLVATVSGSQQTLVVALNSDLANPGQVASGSFSEAVNASNGQVRVWRSGSGDGGGNDGGEGGLVNVNFRCDNGVTQMGDSVYAVGNVSQLGNWSPASAVRLTDTSSYPTWKGSIALPDGQNVEWKCLIRNEADATLVRQWQSGGNNQVQAAAGASTSGSF

SEQ ID NO: 2 - protein, 429 aa, Artificial Sequence
  Synthetic sequence PS4 with signal sequence and starch binding domain removed
DQAGKSPAGVRYHGGDEIILQGFHWNVVREAPNDWYNILRQQASTIAADGFSAIWMPVPWRDFSSWTDGGKSGGGEGYFWHDFNKNGRYGSDAQLRQAAGALGGAGVKVLYDVVPNHMNRGYPDKEINLPAGQGFWRNDCADPGNYPNDCDDGDRFIGGESDLNTGHPQIYGMFRDELANLRSGYGAGGFRFDFVRGYAPERVDSWMSDSADSSFCVGELWKGPSEYPSWDWRNTASWQQIIKDWSDRAKCPVFDFALKERMQNGSVADWKHGLNGNPDPRWREVAVTFVDNHDTGYSPGQNGGQHHWALQDGLIRQAYAYILTSPGTPVVYWSHMYDWGYGDFIRQLIQVRRTAGVRADSAISFHSGYSGLVATVSGSQQTLVVALNSDLANPGQVASGSFSEAVNASNGQVRVWRSGSGDGGGNDGG

SEQ ID NO: 3 - protein, 430 aa, Artificial Sequence
  Synthetic sequence PS4 Variant 3 (SAS3)
MDQAGKSPAGVRYHGGDEIILQGFHWNVVREAPYNWYNILRQQASTIAADGFSAIWMPVPWRDFSSWTDGDKSGGGEGYFWHDFNKNGRYGSDAQLRQAAGALGGAGVKVLYDVVPNHMNRFYPDKEINLPAGQRFWRNDCPDPGNGPNDCDDGDRFLGGEADLNTGHPQIYGMFRDEFTNLRSGYGAGGFRFDFVRGYAPERVDSWMSDSADSSFCVGELWKEPSEYPPWDWRNTASWQQIIKDWSDRAKCPVFDFALKERMQNGSVADWKHGLNGNPDPRWREVAVTFVDNHDTGYSPGQNGGQHKWPLQDGLIRQAYAYILTSPGTPVVYWPHMYDWGYGDFIRQLIQVRRTAGVRADSAISFHSGYSGLVATVSGSQQTLVVALNSDLANPGQVASGSFSEAVNASNGQVRVWRSGSGDGGGNDGG

SEQ ID NO: 4 - DNA, 3050 bp, Pseudomonas saccharophila
  misc_feature: DNA sequence of nucleotide encoding G4-amylase (PS4) from Pseudomonas saccharophila; GenBank Acc. No. X16732
gatcggcgtaggtttcgcattcgttgcccaggcgatatttcgccggtgcgccagcagcctggaagcaggcctggtcgccgccgccggccgtggcgccgacgcccgaacgcagatagccgtggaaatcgaccgccagggccgggccgccgaccagcagggcggcaagcaggcaggcgggttttaggacgaacagggggtgcgcggtgtgcttcatgacgaggtccttgtttttcttgttaatgccgaatcgatcacgccttcgctgcgtgtcgcagggcgcagctcggtggcgaaagcctcggggatggctccgctggcggcatcctcccgaccagagatttcgctggcgcagctcgagggcgtaatcaggatgagtgcggcgtaatccctggggtggggctacgcccggcagggcgcagatgattgccaggggccttcggcctggccactacgccgcctgcaactgggcgggggaggttggtggtcggggcgtgcaggggcagcctgcgggtgccggtcgaagacccggccggcgttcatcctcgtccggcggccttgccgtaggatacccgaacaagcacaagaaccggagtattgcgatgagccacatcctgcgtgccgccgtattggcggcggtcctgctgccgtttcccgcactggccgatcaggccggcaagagcccggccggggtgcgctaccacggcggcgacgaaatcatcctccagggcttccactggaacgtcgtccgcgaagcgcccaacgactggtacaacatcctccgccaacaggcctcgacgatcgcggccgacggcttctcggcaatctggatgccggtgccctggcgtgacttctccagctggaccgacggcggcaagtccggcggcggcgaaggctacttctggcacgacttcaacaagaacggccgctacggcagcgacgcccagctgcgccaggccgccggcgcactcggtggcgccggggtgaaggtgctctacgatgtggtgcccaatcacatgaaccgcggctacccggacaaggagatcaacctgccggccggccagggcttctggcgcaacgactgcgccgacccgggcaactaccccaacgactgcgacgacggtgaccgcttcatcggcggcgagtcggacctgaacaccggccatccgcagatttacggcatgtttcgcgacgagcttgccaacctgcgcagcggctacggcgccggcggcttccgcttcgacttcgttcgcggctatgcgcccgagcgggtcgacagctggatgagcgacagcgccgacagcagcttctgcgttggcgagctgtggaaaggcccttctgaatatccgagctgggactggcgcaacacggcgagctggcagcagatcatcaaggactggtccgaccgggccaagtgcccggtgttcgacttcgctctcaaggagcgcatgcagaacggctcggtcgccgactggaagcatggcctcaatggcaaccccgacccgcgctggcgcgaggtggcggtgaccttcgtcgacaaccacgacaccggctattcgcccgggcagaacggcggccagcaccactgggcgctgcaggacgggctgatccgccaggcctacgcctacatcctcaccagcccgggcacgccggtggtgtactggtcgcacatgtacgactggggctacggcgacttcatccgccagctgatccaggtgcggcgcaccgccggcgtgcgcgccgattcggcgatcagcttccatagcggctacagcggtctggtcgctaccgtcagcggcagccagcagaccctggtggtggcgctcaactccgatctggccaaccccggccaggttgccagcggcagcttcagcgaggcggtcaacgccagcaacggccaggtgcgcgtctggcgcagcggtagcggcgatggcggcgggaatgacggcggcgagggtggcttggtcaatgtgaactttcgctgcgacaacggcgtgacgcagatgggcgacagcgtctacgcggtgggcaacgtcagccagctcggcaactggagcccggcctccgcggtacggctgaccgacaccagcagctatccgacctggaagggcagcatcgccctgcctgacggtcagaacgtggaatggaagtgcctgatccgcaacgaggcggacgcgacgctggtgcgtcagtggcaatcgggcggcaacaaccaggtccaggccgccgccggcgcgagcaccagcggctcgttctgacgacatgcccgcccggcctcggctacgcctacgccgggcggctcctcccgacccagggtgggcagggaggaggccggcgacgggccgggccgccgatgctggcacgacaaccataaaagccttcgcgctgcgctgtcgtatcaggagctgttcatgttggcccagacccgctcgacccctttccggcttggcttcctggcccggctgtacctgctgatcgccgcactggtggccttgctgatgctggtagccggcaccagcctggttgccatcggccgcctgcaaggcaatgccgagcaaatctcgtcgaccgcgtcgcgtctgctggtcagcgagagcttcttcggtacgttgcagagcctgacgcagaacctgtccgacgccctggccgaggaccggcctgaccagctcgacggctatgtcggccggcatcgcacgctgcaggaccaggccctcgagctgttcgcccagctggagcgggtgacgccggcacatgccgagaccaagcaagcctggcggcgctgttgccggagctcgaccgccgcagcctggcgctgatcgatgcgcacgcgacctgctcgcgcgtggggcgcaacgccgtcgcctgcgcgatctgcagctgcagttctcgcggctcaagcaggacctgctgcaggcgcagttcgtgacgggcgacgagctggtcgcctattccatcaagcagttcatcatcccgctcgagcaggtcgagcgctgctgttcgatgccatcggcgtgtcttcgatcaaggcactcgatgaagcgggtgcgcagatc